(19) United States
(12) Patent Application Publication
(10) Pub. No.: US 2023/0270823 A1
(43) Pub. Date: Aug. 31, 2023

(54) LONG-ACTING IL-15 AND USES THEREOF

(71) Applicant: WUXI BIOLOGICS IRELAND LIMITED, Dublin (IE)

(72) Inventors: Yu LIANG, Shanghai (CN); Ming LEI, Shanghai (CN); Jijie GU, Shanghai (CN); Jing LI, Shanghai (CN); Mengmeng SUN, Shanghai (CN); Jing XU, Shanghai (CN); Junqing CUI, Shanghai (CN); Yuanyuan ZHENG, Shanghai (CN); Jianqing XU, Shanghai (CN); Ruipu XIN, Shanghai (CN)

(21) Appl. No.: 18/023,702

(22) PCT Filed: Sep. 1, 2021
(86) PCT No.: PCT/CN2021/115970

§ 371 Date: Feb. 27, 2023

(30) Foreign Application Priority Data
Sep. 1, 2020 (WO) PCT/CN2020/112834

(51) Int. Cl. A61K 38/20; C07K 14/54; C07K 14/715; A61K 45/06

(52) U.S. Cl. CPC A61K 38/2086; C07K 14/5443; C07K 14/7155; A61K 45/06; C07K 2319/30

(57) ABSTRACT

Provided are IL-15 variants and heterodimeric IL-15/IL-15Rα-Fc fusion proteins comprising the IL-15 variants, the methods of producing the same and the uses thereof. The IL-15 variants and fusion proteins of provided can be used as a potent agent for the treatment of cancers.

### CROSS REFERENCE

This application claims priority to International Patent Application No. PCT/CN2020/112834, filed on Sep. 1, 2020, the entire contents of which is incorporated herein by reference.

### SEQUENCE LISTING

The instant application contains a sequence listing which is hereby incorporated by reference in its entirety.

### FIELD

This application generally relates to fusion proteins comprising IL-15. More specifically, the application relates to IL-15-Fc fusion proteins, heterodimeric IL-15/IL-15Rα-Fc fusion proteins, a method for preparing the same, and the uses thereof.

### BACKGROUND

Cancer immunotherapy represented by anti-PD-(L)1 therapy has been approved for many types of cancers and brought in substantial clinical benefits to cancer patients. However, many patients do not respond to the approved PD-(L)1 monotherapy. Cytokine based therapies may overcome such treatment resistance as a mono or combinatorial agent and fulfill the huge unmet medical needs.

However, although recombinant pro-inflammatory cytokines (IL-2, IFNα, etc.) have been approved for cancer treatment for decades, their efficacies are often limited mainly due to short half-life and toxicity. IL-15, an IL-2 family proinflammatory cytokine important for antiviral and anti-tumoral immunity, is involved in the proliferation and survival of many immune cells (especially the cytolytic T/NK cell), without induction of immuno-suppressive Treg and activation-induced cell death in T/NK cells. IL-15 is produced in monocytes and dendritic cells and is primarily presented as a membrane-bound heterodimeric complex with IL-15 receptor α (IL-15Rα) present in the same cells. Its effects are realized through transpresentation of the IL-15/IL-15Rα complex to NK cells and CD8+ T cells expressing IL-2Rβ and common gamma chain.

IL-15 has been tested in various clinical trials. However, its potential in cancer immunotherapy is still greatly limited by the short half-life and toxicity. There remains a need to develop novel IL-15 variants, conjugates, fusion proteins, or complexes to improve treatment efficacy.

The present disclosure provides potency reduced IL-15 variants with enhanced PK/PD profile which may serve as a novel immunotherapy agent with improved anti-tumor efficacy, especially in combination with other treatment options.

### SUMMARY

These and other objectives are provided for by the present disclosure which, in a broad sense, is directed to compounds, methods, compositions and articles of manufacture that provide proteins with improved efficacy. The benefits provided by the present disclosure are broadly applicable in the field of therapeutics and diagnostics and may be used in conjunction with antibodies that react with a variety of targets.

In some aspects, the present disclosure provides an IL-15 variant, wherein the IL-15 variant has a lower potency in stimulating immune cell proliferation compared to wild-type IL-15 protein. In some embodiments, the IL-15 variants have a lower potency in stimulating the proliferation of e.g. NK, CD8+ T cells, and/or CD45+ lymphocytes. In some embodiments, the IL-15 variant has an amino acid sequence comprising one or more substitution(s) compared to SEQ ID No: 1.

In some embodiments, the IL-15 variant as disclosed herein has an amino acid sequence comprising one or more mutation(s) compared to SEQ ID No: 1, wherein the mutation may occur at position 7, 8, 9, 10, 11, 30, 32, 61, 64, 65, 66, 68, 69, 108 and/or 112 of the amino acid sequence as set forth in SEQ ID No: 1. The mutation may be a substitution or insertion of one or more amino acids. Specifically, the one or more mutation(s) may be selected from a group consisting of:
(a) S7N, S7Q, S7A, S7D, S7E, S7F, S7G, S7H, S7I, S7K, S7L, S7M, S7P, S7R, S7T, S7V, S7W, and S7Y substitution;
(b) D8E, D8S, D8T, D8Q, K10Q, D30E, D30Q, H32N, D61Q, and N65Q substitution; (c) I68A, I68V, I68F, I68G, I68K, I68R, I68L, I68M, I68Q, I68D, I68E, I68H, I68N, I68P, I68S, I68T, I68W, and I68Y substitution;
(d) L69A, L69V, L69D, L69E, L69F, L69G, L69H, L69I, L69K, L69M, L69N, L69P, L69Q, L69R, L69S, L69T, L69W, and L69Y substitution;
(e) Q108N, and Ni 12Q substitution;
(f) an insertion in the amino acid sequence of helix A, wherein the amino acid sequence of helix A consists of position 1 to 17 of SEQ ID No: 1; and
(g) an insertion in the amino acid sequence of helix C, wherein the amino acid sequence of helix C consists of amino acids at position 57 to 77 of SEQ ID No: 1.

In some further embodiments, said insertion is a one amino acid insertion that disrupts the hydrogen bonding, salt bridge, and/or van der Waals interaction formed between helix A and IL-2Rβ, helix A and IL-2Rγ, or between helix C and IL-2Rβ. Specifically, the insertion is occurred between I6 and S7, between S7 and D8, between D8 and L9, between L9 and K10, between K10 and K11, between K11 and I12, between H60 and D61, between D61 and T62, between E64 and N65, between N65 and L66, between I67 and I68, between I68 and L69, and/or between L69 and A70.

The inserted amino acid as disclosed herein may comprise non-polar amino acid such as Ala, Gly, Val, Leu, Ile, Met, Trp, Phe, Pro, polar amino acid such as Ser, Thr, Cys, Tyr, Asn, Gln, charged amino acid such as Asp, Glu, Lys, Arg, His, and unnatural amino acid such as a synthetic amino acid.

Preferably, the inserted amino acid is Ala or Gly.

In some embodiments, the IL-15 variant comprises an amino acid sequence comprising one or more substitution(s) selected from a group consisting of: S7N, S7Q, D8E, D8S, D8T, D8Q, K10Q, D30E, D30Q, H32N, D61Q, N65Q, I68A, I68V, I68F, I68G, I68K, I68R, L69A, L69V, Q108N, and N112Q, compared to SEQ ID No: 1.

In some specific embodiments, the IL-15 variant comprises an amino acid sequence comprising one or more substitution(s) selected from a group consisting of: S7N, D8E, D8S, D8T, D8Q, K10Q, D30Q, D61Q, N65Q, I68A, I68F, I68G, I68K, I68R, L69A, L69V, and Q108N. For example, the IL-15 variant may comprise an amino acid sequence comprising one substitution selected from a group consisting of: S7N, D8E, D8S, D8T, D8Q, K10Q, D30Q, D61Q, N65Q, I68A, I68F, I68G, I68K, I68R, L69A, L69V, and Q108N. Alternatively, the IL-15 variant may comprise an amino acid sequence comprising at least two (such as two, three or more) substitutions selected from a group consisting of: S7N, D8E, D8S, D8T, D8Q, K10Q, D30Q, D61Q, N65Q, I68A, I68F, I68G, I68K, I68R, L69A, L69V, and Q108N.

In some embodiments, the IL-15 variant as disclosed herein comprises two or more substitution(s) selected from S7N, K10Q, I68A and L69V. In some embodiments, the IL-15 variant as disclosed herein comprises S7N+K10Q+I68A, K10Q+I68A+L69V, S7N+K10Q+L69V, S7N+I68A+L69V, S7N+K10Q, S7N+I68A, S7N+L69V, K10Q+I68A, K10Q+L69V, or I68A+L69V.

In some embodiments, the potency in stimulating NK and/or CD8+ T cell proliferation is measured in a proliferation assay by FACS. The proliferation assay may use Ki-67 and/or pSTAT5, among others, as a marker or indicator.

In some aspects, the present disclosure provides a fusion protein comprising the IL-15 variant as disclosed herein operably linked to a non-IL-15 moiety. In some embodiments, the non-IL-15 moiety is an antigen-binding domain or an Fc domain.

In some embodiments, the present disclosure provides a fusion protein comprising an IL-15 domain operably linked to a single chain Fc domain, wherein the IL-15 domain comprises the IL-15 variant as disclosed herein. In some embodiments, the single chain Fc domain comprises a human IgG Fc such as a human IgG1 Fc or IgG2 Fc, or a variant thereof. In some embodiments, the IL-15 domain is operably linked to the non-IL-15 moiety (e.g. Fc domain) via a linker, such as a peptide linker, for example (G4S)n with n=1-5. In some embodiments, the linker is GGGGS or (G4S)₂.

In some aspects, the present disclosure provides a heterodimeric fusion protein comprising:
(A) a first chain, comprising an IL-15 domain operably linked to one chain of an Fc domain, wherein the IL-15 domain comprises the IL-15 variant as disclosed herein; and
(B) a second chain, comprising an IL-15Rα domain operably linked to the other chain of the Fc domain.

In some embodiments, the IL-15Rα domain comprises or consists of a wild-type IL-15Rα, an IL-15Rα variant or any fragment thereof that retains IL-15 binding activity. In some embodiments, the IL-15Rα domain comprises or consists of an amino acid sequence as set forth in SEQ ID No: 2.

In some embodiments, the Fc domain comprises a human IgG Fc such as a human IgG1 Fc or IgG2 Fc, or a variant thereof. For example, each chain of the Fc variant may comprise one or more substitutions compared to wild type human Fc to promote heterodimerization. In some specific embodiments, the Fc variant comprises: SEQ ID No: 3 or an amino acid sequence with at least 80% identity to SEQ ID No: 3 for one chain, and SEQ ID No: 4 or an amino acid sequence with at least 80% identity to SEQ ID No: 4 for the other chain.

In some other embodiments, the Fc variant comprises: SEQ ID No: 5 or an amino acid sequence with at least 80% identity to SEQ ID No: 5 for one chain, and SEQ ID No: 6 or an amino acid sequence with at least 80% identity to SEQ ID No: 6 for the other chain.

Still in some other embodiments, the Fc variant comprises: SEQ ID No: 25 or an amino acid sequence with at least 80% identity to SEQ ID No: 25 for one chain, and SEQ ID No: 26 or an amino acid sequence with at least 80% identity to SEQ ID No: 26 for the other chain.

In some embodiments, the Fc variant for constructing the heterodimeric fusion protein as disclosed herein comprises:
(a) a first chain consisting of SEQ ID No: 3, and a second chain consisting of SEQ ID No: 4; or
(b) a first chain consisting of SEQ ID No: 5, and a second chain consisting of SEQ ID No: 6.

In some embodiments, the IL-15 domain and/or IL-15Rα domain is operably linked to the Fc domain via a linker, such as a peptide linker, for example (G4S)n with n=1-5.

In some embodiments, the first chain of the heterodimeric fusion protein as disclosed herein comprises an amino acid sequence as set forth in SEQ ID No: 7, 9, 11, 13, 15, 17, 19, 21, or 23, and/or the second chain comprises an amino acid sequence as set forth in SEQ ID No: 8, 10, 12, 14, 16, 18, 20 or 24.

In some aspects, the present disclosure provides an isolated nucleic acid molecule, comprising a nucleic acid sequence encoding the IL-15 variant or the fusion protein as disclosed herein.

In some aspects, the present disclosure provides a vector comprising the nucleic acid molecule as disclosed herein and a host cell comprising the vector.

In some aspects, the present disclosure provides a pharmaceutical composition comprising the heterodimeric fusion protein as disclosed herein and a pharmaceutically acceptable carrier.

In some aspects, the present disclosure provides an immunoconjugate comprising the IL-variant or the heterodimeric fusion protein as disclosed herein conjugated to an agent. The agent may be a polypeptide, carbohydrate, lipid, nucleic acid or a combination thereof.

In some aspects, the present disclosure provides a method for producing the heterodimeric fusion protein as disclosed herein comprising the steps of:
expressing the heterodimeric fusion protein in the host cell as disclosed herein; and
isolating the heterodimeric fusion protein from the host cell.

In some aspects, the present disclosure provides a method of modulating immune response in a subject, comprising administering the heterodimeric fusion protein or the pharmaceutical composition as disclosed herein to the subject. In some embodiments, the immune response is a NK cell or CD8+ T cell related immune response.

In some aspects, the present disclosure provides a method for treating or preventing cancer in a subject, comprising administering an effective amount of the heterodimeric fusion protein or the pharmaceutical composition as disclosed herein to the subject.

In some embodiments, the method further comprises administering an additional anti-tumor therapy, such as cellular immunotherapy including tumor-infiltrating lymphocyte (TIL) therapy, T cell receptor T cell (TCR-T) therapy, chimeric antigen receptor (CAR) T cell therapy, and NK cell therapy, as well as targeted therapy and chemotherapy. In some specific embodiments, the additional anti-tumor therapy is cellular immunotherapy. The administration of the additional anti-tumor therapy may be performed before, after or simultaneously with the administration of the heterodimeric fusion protein or the pharmaceutical composition as disclosed herein.

The cancer may be a solid tumor cancer or a hematological cancer. In some embodiments, the cancer is selected from e.g. breast cancer, lung cancer, colon cancer, ovarian cancer, melanoma, bladder cancer, renal cell carcinoma, liver cancer, prostate cancer, stomach cancer, pancreatic cancer, NSCLC, non-Hodgkin's lymphoma, chronic lymphocytic leukemia, diffuse large B-cell lymphoma, and multiple myeloma.

In some aspects, the present disclosure provides use of the heterodimeric fusion protein as disclosed herein in the manufacture of a medicament for treating or preventing cancer.

In some aspects, the present disclosure provides the heterodimeric fusion protein as disclosed herein for use in treating or preventing cancer.

In some aspects, the present disclosure provides a kit for treating or diagnosing cancer, comprising a container comprising the heterodimeric fusion protein as disclosed herein.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE FIGURES

FIG. 1a illustrates the structure of a heterodimeric IL-15/Rα-Fc fusion proteins according to some embodiments of the disclosure.

FIG. 1b illustrates the percentage of Ki67 expression in human (a) CD8+ T cells and (b) NK cells following treatment with WT IL-15/Ru fused with two different Fc formats.

FIG. 2 illustrates the percentage of Ki67 expression in human (a, c, e) CD8+ T cells and (b, d, f) NK cells following treatment with indicated IL-15/Rα-Fc fusion proteins.

FIG. 3 illustrates STAT5 phosphorylation in NK cells (CD3− CD56+) following incubation of PBMCs for 30 minutes with the indicated variant IL15/Rα-Fc fusion proteins.

FIG. 4 illustrates the percentage of Ki67 expression in mouse (a, c) CD8+ T cells and (b, d) NK cells following treatment with indicated IL-15/Rα-Fc fusion proteins.

FIGS. 5-6 illustrate the percentage of Ki67 expression in human (a) CD8+ T cells and (b) NK cells following treatment with indicated IL-15/Rα-Fc fusion proteins.

FIG. 7 illustrates the percentage of Ki67 expression in mouse (a) CD8+ T cells and (b) NK cells following treatment with indicated IL-15/Rα-Fc fusion proteins.

FIG. 8 illustrates the percentage of Ki67 expression in human (a) CD8+ T cells and (b) NK cells following treatment with indicated IL-15/Rα-Fc fusion proteins in V47 format.

FIG. 9 illustrates the percentage of Ki67 expression in cyno (a) CD8+ T cells and (b) NK cells following treatment with indicated IL-15/Rα-Fc fusion proteins in V47 format.

FIG. 10 illustrates the serum concentration of test articles in cynomolgus monkeys after intravenous injection.

FIG. 11 illustrates pharmacodynamics effects measured by fold change in cell counts of total CD45+ Lymphocytes (a), CD8+ T cells (b) and NK cells (c), in cyno monkey peripheral blood upon treatment with the test articles.

FIG. 12 illustrates pharmacodynamics effect measured by % of Ki-67 proliferation marker in CD8+ T cells (a) and NK cells (b), in cyno monkey peripheral blood upon treatment with the test articles.

FIG. 13 illustrates Th1 (IL-2, TNF, IFN-γ) and Th2 (IL-4, IL-5, IL-6) inflammatory cytokine level in cyno monkey peripheral blood upon treatment.

FIG. 14 illustrates serum albumin levels after treatment with indicated IL-15/Rα-Fc fusion proteins.

FIG. 15 illustrates clinical responses to IL-15 treatment: temperature (a), body weight (b), mean arterial pressure (MAP; c), shown by individual monkey. “V0006” and “V0061” represent W369-E17-TxU1-V0006.uIgG1V47 and W369-E17-TxU1-V0061.uIgG1V47 treatment, respectively.

FIG. 16 illustrates the blood routine examination after IL-15 molecule infusions. White blood cells (WBCs; a), lymphocytes (b), monocytes (c), basophiles (BASO; d) and neutrophils (NEUT; e) are shown by individual monkey.

FIG. 17 illustrates the percentage of Ki67 expression on human (a) CD8+ T cells and (b) NK cells following treatment with IL-15/Ra variants in V805 format.

FIG. 18 illustrates the percentage of Ki67 expression on cyno CD8+ T cells (a,b) and NK cells (c, d) following treatment with IL-15/Ra variants in V805 format.

FIG. 19 illustrates crystal structure of IL-15 and possible poses of IL-15 variants with receptors: (A). Crystal structure of wild type IL-15 interacting with its receptors. Key interactions were highlighted: two hydrogen bonds between D8 (IL-15 Helix A) and H133/Y134 (receptor β), and one hydrogen bond between S7 (IL-15 Helix A) and E135 (receptor β); (B). In silico model of IL-15 variant v0001 (Ala insertion between S7 and D8). Ala residue was inserted in IL-15 Helix A to interrupt the interactions with its receptors: the Ala insertion at site 8 could potentially disrupt the original two hydrogen bonds formed between D8 (IL-15 Helix A) and H133/Y134 (receptor β), and weaken the original hydrogen bond between S7 (IL-15 Helix A) and E135 (receptor β); (C). Crystal structure of wild type IL-15 interacting with its receptors. Key interactions were highlighted: three hydrogen bonds between N64 (IL-15 Helix C) and R42/Q70 (receptor 3) and one salt bridge between E65 (IL-15 Helix C) and R42 (receptor β); (D). In silico model of IL-15 variant v0005 (Ala insertion between E64 and N65). The Ala insertion at site 65 could potentially affect the position of N64 and E65 (IL-15 Helix) and break all their original interactions with R42 and Q70 (receptor (3).

### DETAILED DESCRIPTION

While the present disclosure may be embodied in many different forms, disclosed herein are specific illustrative embodiments thereof that exemplify the principles of the disclosure. It should be emphasized that the present disclosure is not limited to the specific embodiments illustrated. Moreover, any section headings used herein are for organizational purposes only and are not to be construed as limiting the subject matter described.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like. In this application, the use of “or” means “and/or” unless stated otherwise. Furthermore, the use of the term “comprising,” as well as other forms, such as “comprises” and “comprised,” is not limiting. In addition, ranges provided in the specification and appended claims include both end points and all points between the end points.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. See, e.g., Abbas et al., Cellular and Molecular Immunology, 6^{th} ed., W.B. Saunders Company (2010); Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual, Cold Spring Harbor Laboratory Press, Cold Spring Harbor, N.Y. (1998); and Coligan et al., Short Protocols in Protein Science, Wiley, John & Sons, Inc. (2003). The nomenclature used in connection with, and the laboratory procedures and techniques of, analytical chemistry, synthetic organic chemistry, and medicinal and pharmaceutical chemistry described herein are those well-known and commonly used in the art.

### Definitions

In order to better understand the disclosure, the definitions and explanations of the relevant terms are provided as follows.

The term “IL-15”, as used herein, refers to Interleukin-15 and is intended to encompass any form of IL-15, for example, 1) native unprocessed IL-15 molecule, “full-length” IL-15 protein or naturally occurring variants of IL-15; 2) any form of IL-15 that results from processing in the cell; or 3) full length or a modified form. A wild-type IL-15 is a 14-15 kDa member of the four-α-helix bundle family of cytokines that is involved in natural killer (NK) cell differentiation, T-cell functions, and the host response to intracellular pathogens. An example of the amino acid sequence of human wild-type IL-15 is shown in SEQ ID No: 1. As used in the disclosure, the term “IL-15” or “IL-15 domain” includes both wild-type IL-15 and IL-15 variants.

The term “IL-15Rα” or “IL-15 receptor α”, as used herein, refers to a high-affinity receptor of IL-15, which binds to IL-15 and transduces signals in the presence of the IL-15Rβ and γc (common γ chain). The full-length human IL-15Rα is a type-1 transmembrane protein with a signal peptide, an extracellular domain, a transmembrane domain and a cytoplasmic tail. As used in the disclosure, the term “IL-15Rα” or “IL-15Rα domain” includes both wild-type IL-15Rα and IL-15Rα variants and fragments thereof. As is known in the art, the IL-15Rα protein contains a “sushi domain”, which is the shortest region of the receptor that retains IL-15 binding activity (as shown in SEQ ID No: 2). In some embodiments, the “sushi domain”, i.e. the peptide as shown in SEQ ID No: 2 is used to construct the IL-15/Rα-Fc fusion proteins.

The term “variant”, with regard to polypeptide or protein, means a biologically active polypeptide which includes one or more amino acid mutations in the native protein sequence. Optionally, the one or more amino acid mutations include amino acid substitution and/or insertion at certain positions within the amino acid sequence. Preferably, a variant has at least about 80% amino acid sequence identity with the corresponding native sequence polypeptide. Such variants include, for instance, polypeptides wherein one or more amino acid (naturally occurring amino acid and/or a non-naturally occurring amino acid) residues are added at the N- and/or C-terminus of the polypeptide. Variants thereof for use in the disclosure can be prepared by a variety of methods well known in the art, such as site-directed mutagenesis of nucleotides in the DNA encoding the native protein or phage display techniques, thereby producing DNA encoding the variant, and thereafter expressing the DNA in recombinant cell culture. In certain embodiments as disclosed herein, an IL-15 variant comprises one or more substitutions compared to the wild-type IL-15 protein.

The term “Fc”, as used herein, has a same meaning as used with regard to an antibody, which refers to that portion of the antibody comprising the second (CH2) and third (CH3) constant regions of a first heavy chain bound to the second and third constant regions of a second heavy chain via disulfide bonding. The Fc region may also comprise part or whole of the hinge region. The Fc region of the antibody is responsible for various effector functions such as ADCC and CDC, but does not function in antigen binding. In the present disclosure, the term “Fc” includes both wild-type Fc and Fc variants.

The term “modification”, with respect to an amino acid residue/position as used herein, refers to a change of a primary amino acid sequence as compared to a starting amino acid sequence, wherein the change results from a sequence alteration involving said amino acid residue/positions. For example, typical modifications include substitution of the residue (or at said position) with another amino acid (e.g., a conservative or non-conservative substitution), and insertion of one or more amino acids adjacent to said residue/position. An “amino acid substitution”, or variation thereof, refers to the replacement of an existing amino acid residue in a predetermined (starting) amino acid sequence with a different amino acid residue. Generally, the modification results in alteration in at least one physicobiochemical activity of the variant polypeptide compared to a polypeptide comprising the starting (or “wild type”) amino acid sequence. For example, in an IL-15 variant, a physicobiochemical activity that is altered can be binding affinity, binding capability and/or binding effect upon a target molecule. As used herein, two or more substitutions in an amino acid sequence may be expressed with “+” or “/” between each substitution. For example, a double substitution like S7N and K10Q may be expressed as “S7N/K10Q” or “S7N+K10Q”, etc.

The term “fusion protein”, as used herein, refers to a polypeptide having two (or more) portions operably linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. In certain embodiments, the two portions of the fusion protein are IL-15 protein domain and Fc domain, or IL-15Rα protein domain and Fc domain. The two fusion proteins may be associated together to form a heterodimer. In a broad sense, when mentioning “fusion protein” in the present specification, depending on the context, it may refer to a single chain IL-15-comprising fusion protein or a heterodimeric fusion protein.

As used herein, the term “heterodimer”, “heterodimeric fusion protein”, “heterodimeric IL-15 fusion protein” or “IL-15/Rα-Fc fusion protein” may be used interchangeably, which refers to a heterodimer protein comprising two chains, each chain comprising a polypeptide of an Fc domain, the IL-15 domain may be on the same or other chain with the IL-15Rα domain. In some embodiments, the first chain is a fusion protein of IL-15 protein domain and Fc domain, the second chain is a fusion protein of IL-15Rα protein domain and Fc domain. Such heterodimeric fusion proteins are structurally similar to an antibody, in view that the Fab part in an antibody is replaced by an IL-15/IL-15Rα complex formed by interaction between the IL-15 and IL-15Rα domains.

The terms “operably linked” refer to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “EC₅₀,” as used herein, which is also termed as “half maximal effective concentration” refers to the concentration of a drug, antibody or toxicant which induces a response halfway between the baseline and maximum after a specified exposure time. In the context of the application, EC₅₀ is expressed in the unit of “nM”.

The term “isolated,” as used herein, refers to a state obtained from natural state by artificial means. If a certain “isolated” substance or component is present in nature, it is possible because its natural environment changes, or the substance is isolated from natural environment, or both. For example, a certain un-isolated polynucleotide or polypeptide naturally exists in a certain living animal body, and the same polynucleotide or polypeptide with a high purity isolated from such a natural state is called isolated polynucleotide or polypeptide. The term “isolated” excludes neither the mixed artificial or synthesized substance nor other impure substances that do not affect the activity of the isolated substance.

The term “vector,” as used herein, refers to a nucleic acid vehicle which can have a polynucleotide inserted therein. When the vector allows for the expression of the protein encoded by the polynucleotide inserted therein, the vector is called an expression vector. The vector can have the carried genetic material elements expressed in a host cell by transformation, transduction, or transfection into the host cell. Vectors are well known by a person skilled in the art, including, but not limited to plasmids, phages, cosmids, artificial chromosome such as yeast artificial chromosome (YAC), bacterial artificial chromosome (BAC) or P1-derived artificial chromosome (PAC); phage such as X phage or M13 phage and animal virus. The animal viruses that can be used as vectors, include, but are not limited to, retrovirus (including lentivirus), adenovirus, adeno-associated virus, herpes virus (such as herpes simplex virus), pox virus, baculovirus, papillomavirus, papova virus (such as SV40). A vector may comprise multiple elements for controlling expression, including, but not limited to, a promoter sequence, a transcription initiation sequence, an enhancer sequence, a selection element and a reporter gene. In addition, a vector may comprise origin of replication.

The term “host cell,” as used herein, refers to a cellular system which can be engineered to generate proteins, protein fragments, or peptides of interest. Host cells include, without limitation, cultured cells, e.g., mammalian cultured cells derived from rodents (rats, mice, guinea pigs, or hamsters) such as CHO, BHK, NSO, SP2/0, YB2/0; or human tissues or hybridoma cells, yeast cells, and insect cells, and cells comprised within a transgenic animal or cultured tissue. The term encompasses not only the particular subject cell but also the progeny of such a cell. Because certain modifications may occur in succeeding generations due to either mutation or environmental influences, such progeny may not be identical to the parent cell, but are still included within the scope of the term “host cell”.

The term “identity,” as used herein, refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al, 1988, SIAMJ. Applied Math. 48:1073.

The term “transfection,” as used herein, refers to the process by which nucleic acids are introduced into eukaryotic cells, particularly mammalian cells. Protocols and techniques for transfection include but not limited to lipid transfection and chemical and physical methods such as electroporation. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al, 1981, Gene 13:197. In a specific embodiment of the disclosure, human IL-15 gene was transfected into 293F cells.

The term “fluorescence-activated cell sorting” or “FACS,” as used herein, refers to a specialized type of flow cytometry. It provides a method for sorting a heterogeneous mixture of biological cells into two or more containers, one cell at a time, based upon the specific light scattering and fluorescent characteristics of each cell (FlowMetric. “Sorting Out Fluorescence Activated Cell Sorting”. Retrieved 2017 Nov. 9). Instruments for carrying out FACS are known to those of skill in the art and are commercially available to the public. Examples of such instruments include FACS Star Plus, FACScan and FACSort instruments from Becton Dickinson (Foster City, Calif.) Epics C from Coulter Epics Division (Hialeah, Fla.) and MoFlo from Cytomation (Colorado Springs, Colo.).

The term “antibody-dependent cell-mediated cytotoxicity” or “ADCC,” as used herein, refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The term “subject” includes any human or nonhuman animal, preferably humans.

The term “cancer,” as used herein, refers to any or a tumor or a malignant cell growth, proliferation or metastasis-mediated, solid tumors and non-solid tumors such as leukemia and initiate a medical condition.

The term “treatment,” “treating” or “treated,” as used herein in the context of treating a condition, pertains generally to treatment and therapy, whether of a human or an animal, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. For cancer, “treating” may refer to dampen or slow the tumor or malignant cell growth, proliferation, or metastasis, or some combination thereof. For tumors, “treatment” includes removal of all or part of the tumor, inhibiting or slowing tumor growth and metastasis, preventing or delaying the development of a tumor, or some combination thereof.

The term “an effective amount,” as used herein, pertains to that amount of an active compound, or a material, composition or dosage form comprising an active compound, which is effective for producing some desired therapeutic effect, commensurate with a reasonable benefit/risk ratio, when administered in accordance with a desired treatment regimen. For instance, the “an effective amount,” when used in connection with treatment of diseases or conditions such as cancers, refers to an active agent, a drug or an antibody or antigen-binding portion thereof in an amount or concentration effective to treat the said diseases or conditions.

The term “prevent,” “prevention” or “preventing,” as used herein, with reference to a certain disease condition in a mammal, refers to preventing or delaying the onset of the disease, or preventing the manifestation of clinical or subclinical symptoms thereof.

The term “pharmaceutically acceptable,” as used herein, means that the vehicle, diluent, excipient and/or salts thereof, are chemically and/or physically is compatible with other ingredients in the formulation, and the physiologically compatible with the recipient.

As used herein, the term “a pharmaceutically acceptable carrier and/or excipient” refers to a carrier and/or excipient pharmacologically and/or physiologically compatible with a subject and an active agent, which is well known in the art (see, e.g., Remington's Pharmaceutical Sciences. Edited by Gennaro A R, 19th ed. Pennsylvania: Mack Publishing Company, 1995), and includes, but is not limited to pH adjuster, surfactant, adjuvant and ionic strength enhancer. For example, the pH adjuster includes, but is not limited to, phosphate buffer; the surfactant includes, but is not limited to, cationic, anionic, or non-ionic surfactant, e.g., Tween-80; the ionic strength enhancer includes, but is not limited to, sodium chloride.

As used herein, the term “adjuvant” refers to a non-specific immunopotentiator, which can enhance immune response to an antigen or change the type of immune response in an organism when it is delivered together with the antigen to the organism or is delivered to the organism in advance. There are a variety of adjuvants, including, but not limited to, aluminium adjuvants (for example, aluminum hydroxide), Freund's adjuvants (for example, Freund's complete adjuvant and Freund's incomplete adjuvant), Corynebacterium parvum, lipopolysaccharide, cytokines, and the like. Freund's adjuvant is the most commonly used adjuvant in animal experiments now. Aluminum hydroxide adjuvant is more commonly used in clinical trials.

IL-15, IL-15Rα Protein Domains and IL-15/IL-15Rα Complex

In some aspects, the present disclosure provides IL-15 variants which comprise one or more modification(s), e.g. insertion and/or substitution, compared to the wild-type IL-15 protein, such as human wild-type IL-15 protein. The present disclosure further provides IL-15-Fc fusion proteins comprising IL15 protein domain fused to a Fc domain. The IL15 protein domain may comprise a wild-type IL-15 protein as set forth in SEQ ID No: 1, or an IL-15 variant comprising one or more modification(s), e.g. insertion and/or substitution, compared to the wild-type IL-15 protein.

Residues are designated herein by the one letter amino acid code followed by the IL-15 amino acid position, e.g., S7 is the serine residue at position 7 of SEQ ID NO: 1. Substitutions are designated herein by the one letter amino acid code followed by the IL-15 amino acid position followed by the substituting one letter amino acid code, e.g., S7N is a substitution of the serine residue at position 7 of SEQ ID NO: 1 with a asparagine residue.

In some embodiments, the IL-15 variant has at least one amino acid substitution or at least one amino acid insertion compared to SEQ ID No: 1. In some embodiments, the substitution(s) or insertion(s) occur at position 7, 8, 9, 10, 11, 30, 32, 61, 64, 65, 66, 68, 69, 108 and/or 112 of the amino acid sequence as set forth in SEQ ID No: 1. In some embodiments, the substitution(s) occur at the amino acids located at the interaction interface between IL-15 and IL-15R β and γ chain, such as positions 7, 8, 10, 30, 61, 65, 68, 69, 108, 112 corresponding to SEQ ID No: 1. In some other embodiments, the insertion(s) occur at positions located at the interaction interface between IL-15 helix A and IL-2Rβ/γ, or between helix C and IL-2Rβ, such that the insertion is capable of disrupting the hydrogen bonding, salt bridge, and/or van der Waals interaction. The inventors have identified some of the positions which may be critical for interaction with the receptors, such as positions 7, 8, 10, 11, 61, 65, 68 and 69 corresponding to SEQ ID NO: 1. The insertion thus may be selected from the following: between I6 and S7, between S7 and D8, between D8 and L9, between L9 and K10, between K10 and K11, between K11 and I12, between H60 and D61, between D61 and T62, between E64 and N65, between N65 and L66, between I67 and I68, between I68 and L69, and/or between L69 and A70, particularly, between S7 and D8, between D8 and L9, between L9 and K10, between K10 and K11, between E64 and N65, and between N65 and L66, according to the amino acid sequence as set forth in SEQ ID No: 1. Preferably, the insertion is a one amino acid insertion and the inserted amino acid may comprise non-polar amino acid such as Ala, Gly, Val, Leu, Ile, Met, Trp, Phe, Pro, polar amino acid such as Ser, Thr, Cys, Tyr, Asn, Gln, charged amino acid such as Asp, Glu, Lys, Arg, His, and unnatural amino acid.

In some embodiments, the inserted amino acid is Ala, gly or Ser. By introducing a modification(s) into the amino acid sequence, the IL-15 variants as disclosed herein provide a reduced binding affinity to IL-15Rβ/γ chain, resulting in reduced potency in stimulating immune cells and thus reduced toxicity in vivo.

In some embodiments, the IL-15 variant comprises one or more substitutions (such as 11, 10, 9, 8, 7, 6, 5, 4, 3, or 2 substitutions) at positions 7, 8, 10, 30, 32, 61, 65, 68, 69, 108 and/or 112 of the amino acid sequence as set forth in SEQ ID No: 1. At each position, the amino acid can be substituted by any amino acid other than cysteine and the original amino acid. For example, in some embodiments, the IL-15 variant comprises a substitution at position 7 of the amino acid sequence as set forth in SEQ ID No: 1, the original amino acid serine (S) can be substituted by any amino acid other than cysteine and serine, such as alanine (A), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), threonine (T), valine (V), tryptophan (W), tyrosine (Y). In some embodiments, the IL-15 variant comprises a substitution at position 68 of the amino acid sequence as set forth in SEQ ID No: 1, the original amino acid isoleucine (I) can be substituted by any amino acid other than cysteine and isoleucine, such as A, D, E, F, G, H, K, L, M, N, P, Q, R, S, T, V, W, Y. In some embodiments, the IL-15 variant comprises a substitution at position 69 of the amino acid sequence as set forth in SEQ ID No: 1, the original amino acid leucine (L) can be substituted by any amino acid other than cysteine and leucine, such as A, D, E, F, G, H, I, K, M, N, P, Q, R, S, T, V, W, Y.

In some embodiments, the IL-15 variant comprises a substitution at position 7 and a substitution(s) at position 10, 68 and/or 69. In some embodiments, the IL-15 variant comprises a substitution at position 10 and a substitution(s) at position 68 and/or 69. In some embodiments, the IL-15 variant comprises a substitution at position 68 and/or a substitution at position 69.

In some embodiments, the IL-15 variant comprises one or more substitution(s) selected from a group comprising:
(a) S7N, S7Q, S7A, S7D, S7E, S7F, S7G, S7H, S7I, S7K, S7L, S7M, S7P, S7R, S7T, S7V, S7W, S7Y;
(b) D8E, D8S, D8T, D8Q, K10Q, D30E, D30Q, H32N, D61Q, N65Q;
(c) I68A, I68V, I68F, I68G, I68K, I68R, I68L, I68M, I68Q, I68D, I68E, I68H, I68N, I68P, I68S, I68T, I68W, I68Y;
(d) L69A, L69V, L69D, L69E, L69F, L69G, L69H, L69I, L69K, L69M, L69N, L69P, L69Q, L69R, L69S, L69T, L69W, L69Y;
(e) Q108N, N112Q; and
(f) any combination thereof,

Compared to SEQ ID No: 1.

In some embodiments, the IL-15 variant comprises one or more insertion(s) selected from a group comprising:

(a) an insertion between S7 and D8; (b) an insertion between D8 and L9; (c) an insertion between L9 and K10; (d) an insertion between K10 and K11; (e) an insertion between E64 and N65; and (f) an insertion between N65 and L66; compared to SEQ ID No: 1. Preferably, the inserted amino acid is Ala.

In some embodiments, the IL-15 variant comprises at least two substitutions (e.g. two, three, four, five or even more) selected from a group comprising: S7N, S7Q, D8E, D8S, D8T, D8Q, K10Q, D30E, D30Q, H32N, D61Q, N65Q, I68A, I68V, I68F, I68G, I68K, I68R, I68L, I68M, I68Q, L69A, L69V, Q108N and N112Q.

In some specific embodiments, the IL-15 variant comprises one of the following substitutions: S7N+K10Q+I68A, K10Q+I68A+L69V, S7N+K10Q+L69V, S7N+I68A+L69V, S7N+K10Q, S7N+I68A, S7N+L69V, K10Q+I68A, K10Q+L69V, and I68A+L69V.

The IL-15Rα protein domain may comprise a wild-type IL-15Rα protein, an IL-15Rα variant or any fragment thereof that retains the IL-15 binding activity. In some embodiments, the IL-15Rα protein domain comprises the wild-type IL-15Rα protein or an IL-15 binding fragment thereof as set forth in SEQ ID No: 2. In some embodiments, the IL-15Rα variant has one or more modification(s), e.g. insertion, substitution and/or deletion, compared to SEQ ID No: 2.

The IL-15 protein domain may associate with the IL-15Rα protein domain in different ways to form an IL-15R/IL-15Rα complex. In some embodiments, the IL-15 protein domain and the IL-15Rα protein domain are self-assembled through ligand-ligand interaction, i.e. the binding affinity between the two domains. In some other embodiments, the two domains are covalently linked via a linker, preferably a peptide linker. In some further embodiments, both domains are engineered to include a cysteine amino acid(s) such that a disulfide bond(s) is/are formed between the two domains.

Fc Domain

As described above, single chain IL15-Fc fusion protein and IL15Rα-Fc protein each comprises one chain of a Fc domain, which associates together to form the Fc domain, similar to the Fc region in an antibody.

The Fe domain may be a wild-type Fc or an Fc variant. A wild-type Fc may be a human IgG1, IgG2, IgG3 or IgG4 Fc. In some embodiments, the wild-type Fc is a human IgG1 Fc.

The Fc variant comprises one or more amino acid residue modifications (e.g. substitutions, insertions and/or deletions) compared to the wild-type Fc (e.g, human IgG1, IgG2, IgG3 or IgG4 Fc). In some embodiments, the Fc variant comprises one or more amino acid residue modifications (e.g. substitutions, insertions and/or deletions) compared to wild-type human IgG1 Fc.

Said one or more amino acid modifications comprised in the Fc variant may alter the binding to one or more FcγR receptors, alter the binding to FcRn receptors, etc. In certain embodiments, the Fc domain of the fusion proteins comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody and antigen-binding fragment thereof to improve binding affinity with FcRn are well-known in the art, see, for example, Vaughn, D. et al, Structure, 6(1): 63-73, 1998; Kontermann, R. et al, Antibody Engineering, Volume 1, Chapter 27: Engineering of the Fc region for improved PK, published by Springer, 2010; Yeung, Y. et al, Cancer Research, 70: 3269-3277 (2010); and Hinton, P. et al, J. Immunology, 176:346-356 (2006).

The two chains of the Fc domain may associate together via a disulfide bond. In some embodiments, the Fe domain comprises one or more amino acid modifications (e.g. substitutions) in the interface of the Fc region to facilitate and/or promote heterodimerization. For example, the two chains of the Fc domain are engineered to comprise a “knob-into-hole” structure to promote heterodimerization, which includes introduction of a protuberance (“knob”) into a first Fc polypeptide and a cavity (“hole”) into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168. Specifically, the Fc domain may comprise at least one “knob” (protuberance) and at least one “hole” (cavity), wherein presence of the “knob” and “hole” enhances formation of a complex or heterodimer (for more detail see WO 2005/063816). In some embodiments, the Fc domain as disclosed herein comprises a first and a second Fc polypeptide chain, wherein the first and second polypeptide each comprises one or more mutations with respect to wild type human IgG1 Fc. The IL-15 domain may be fused to one chain of the Fc domain comprising a “knob” mutation while the IL-15Rα domain is fused to the other chain comprising a “hole” mutation, or vice versa. In at least one embodiment, a “hole” mutation is Y349C, T366S, L368A, and/or Y407V, and a “knob” mutation is S354C and/or T366W.

In certain embodiments, the Fe domain of the fusion proteins comprise one or more amino acid substitution(s) that alters the antibody-dependent cellular cytotoxicity (ADCC) and/or complement dependent cytotoxicity (CDC). Certain amino acid residues at CH2 domain of the Fc region can be substituted to provide for reduced ADCC activity.

In certain embodiments, the Fe domain further comprises a triple mutation M252Y/S254T/T256E (“YTE”). This triple mutation has been reported to cause an about 10-fold increase in binding to human neonatal Fc receptor (FcRn) and an almost 4-fold increase in the serum half-life of YTE-containing human IgGs in cynomolgus monkeys (Oganesyan V. et al, Mol Immunol. 2009 May; 46(8-9):1750-5). In some further embodiments, the Fe domain comprises additional mutations to enhance the interaction between Fe and human FcRn. In some specific embodiments, the Fe domain comprises C220S, G236R, and/or L328R substitution(s) in addition to the “YTE” triple mutation. A double “G236R/L328R” or “RR” substitution has been reported to reduce binding affinity to FcγR and the Fe effector function (ADCC, CDC, ADCP).

The “EU numbering system” or “EU index” is generally used when referring to a residue in an immunoglobulin heavy chain constant region (e.g., the EU index reported in Kabat et al. Sequences of Proteins of Immunological Interest (5^{th} Ed.), US Dept. of Health and Human Services, PHS, NIH, NIH Publication no. 91-3242). The “EU numbering as in Kabat” or “EU index as in Kabat” refers to the residue numbering of the human IgG1 EU antibody. Unless stated otherwise herein, references to residue numbers in the constant domain of Fc regions means residue numbering by the EU numbering system.

In some specific embodiments, the Fe variant comprises two chains, wherein the amino acid sequence of the first chain has at least 80%, e.g 80%, 85%, 90%, 95% or more (e.g. 100%) sequence identity to SEQ ID No: 3, and/or the amino acid sequence of the second chain has at least 80%, e.g 80%, 85%, 90%, 95% or more (e.g. 100%) sequence identity to SEQ ID No: 4. In some specific embodiments, the first chain of the Fe variant comprises or consists of an amino acid sequence as set forth in SEQ ID No: 3, and the second chain of the Fe variant comprises or consists of an amino acid sequence as set forth in SEQ ID No: 4.

In some specific embodiments, the Fc variant comprises two chains, wherein the amino acid sequence of the first chain has at least 80%, e.g 80%, 85%, 90%, 95% or more (e.g. 100%) sequence identity to SEQ ID No: 5, and/or the amino acid sequence of the second chain has at least 80%, e.g 80%, 85%, 90%, 95% or more (e.g. 100%) sequence identity to SEQ ID No: 6. In some specific embodiments, the first chain of the Fe variant comprises or consists of an amino acid sequence as set forth in SEQ ID No: 5, and the second chain of the Fe variant comprises or consists of an amino acid sequence as set forth in SEQ ID No: 6.

The expression of “at least 80%” with regard to sequence identity may include e.g. 80%, 85%, 88%, 90%, 91%, 92%, 93%, 94%, 95%, 96%, 97%, 98%, 99%, 100% (i.e. the same). The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percentage of identity between two amino acid sequences can be determined by the algorithm of Needleman and Wunsch (J. Mol. Biol. 48:444-453 (1970)) which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally, or alternatively, the protein sequences of the present disclosure can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the fusion protein molecules of the disclosure. To obtain gapped alignments for comparison purposes, Gapped BLAST can be utilized as described in Altschul et al, (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

IL-15-Fc Fusion Proteins and Heterodimeric IL-15/IL-15Rα-Fc Fusion Proteins

In some aspects, the present disclosure provides single chain IL-15-Fc fusion proteins that comprise IL-15 protein domains and an Fc domain, and IL-15Rα-Fc fusion proteins that comprise IL-15Rα protein domains and an Fc domain. Further, the two fusion proteins may heterodimerize to form an IL-15/IL-15Rα-Fc fusion protein comprising two chains. Each of the IL-15 domain, IL-15Rα domain and Fc domain constituting the heterodimeric IL-15/IL-15Rα-Fc fusion protein can be in wild-type format or corresponding variants, as described above.

As used herein, the term “long acting” means an IL-15-Fc fusion protein (monomer or heterodimeric) comprising an IL-15 variant operably linked to an immunoglobulin Fc region that provides a prolonged pharmacokinetics (PK) profile, as shown in e.g. a longer serum half-life, increased Cmax, lower serum clearance and improved drug exposure (AUC), or a pronounced and extended pharmacodynamics (PD) effect on lymphocyte expansion, especially proliferation of NK and CD8+ T cells.

In some embodiments, the heterodimeric IL-15/IL-15Rα-Fc fusion proteins as disclosed herein comprise two chains, the first chain comprises a fusion of the IL-15 domain with one chain of the Fc domain, the second chain comprises a fusion of the IL-15Rα domain with the other chain of the Fc domain. In some embodiments, the IL-15/IL-15Rα domain is operably linked to the Fc domain. Preferably, the IL-15/IL-15Rα domain is operably linked to the N terminus of the Fc domain.

In some other embodiments, the IL-15/IL-15Rα-Fc fusion proteins as disclosed herein comprise two chains, the first chain comprises a fusion of the IL-15 domain and IL-15Rα domain with one chain of the Fc domain, the second chain comprises the other chain of the Fc domain. For example, the IL-15 domain may be operably linked to the IL-15Rα domain (optionally via a linker), and further operably linked to one chain of the Fc domain, such as IL15-IL15Rα-Fc or IL15Rα-IL15-Fc from N terminal to C terminal, wherein “-” represents an operable linkage.

In some embodiments, the IL-15/IL-15Rα domain is directly linked to the Fc domain, i.e. without a linker; more often, the IL-15/IL-15Rα domain is linked to the Fc domain via a linker, such as a peptide linker with one or more amino acid residues. The linker should provide sufficient flexibility to allow two domains at each end to perform their respective function. Many suitable linkers are available in the art and can be used here. In some embodiments, the linker is (G4S)n, wherein n may be 1-5 (such as 1, 2, 3, 4 or 5).

In some embodiments, the IL-15/IL-15Rα-Fc fusion proteins as disclosed herein comprise two chains, the first chain comprises an IL-15 domain operably linked to one chain of an IgG1 Fc domain, the second chain comprises a IL-15Rα protein domain operably linked to the other chain of the IgG1 Fc domain, and the IL-15 domain comprises an IL-15 variant as described above. In some exemplary embodiments, the IL-15 variant comprises one or more mutation(s) selected from a group consisting of:
(a) S7N, S7Q, S7A, S7D, S7E, S7F, S7G, S7H, S7I, S7K, S7L, S7M, S7P, S7R, S7T, S7V, S7W, and S7Y substitution;
(b) D8E, D8S, D8T, D8Q, K10Q, D30E, D30Q, H32N, D61Q, and N65Q substitution; (c) I68A, I68V, I68F, I68G, I68K, I68R, I68L, I68M, I68Q, I68D, I68E, I68H, I68N, I68P, I68S, I68T, I68W, and I68Y substitution;
(d) L69A, L69V, L69D, L69E, L69F, L69G, L69H, L69I, L69K, L69M, L69N, L69P, L69Q, L69R, L69S, L69T, L69W, and L69Y substitution;
(e) Q108N and N112Q substitution;
(f) an insertion in the amino acid sequence of helix A, wherein the amino acid sequence of helix A consists of position 1 to 17 of SEQ ID No: 1 (for example, between S7 and D8, between D8 and L9, between L9 and K10, between K10 and K11); and
(g) an insertion in the amino acid sequence of helix C, wherein the amino acid sequence of helix C consists of amino acids at position 57 to 77 of SEQ ID No: 1 (for example, between E64 and N65, and between N65 and L66); and
(h) any combination thereof.

In some specific embodiments, the IL-15 domain comprises an IL-15 variant selected from a group consisting of:

an IL-15 variant comprising a S7N substitution compared to SEQ ID No: 1; an IL-15 variant comprising a D8E substitution compared to SEQ ID No: 1; an IL-15 variant comprising a D8S substitution compared to SEQ ID No: 1; an IL-15 variant comprising a D8T substitution compared to SEQ ID No: 1; an IL-15 variant comprising a D8Q substitution compared to SEQ ID No: 1; an IL-15 variant comprising a K10Q substitution compared to SEQ ID No: 1; an IL-15 variant comprising a D30Q substitution compared to SEQ ID No: 1; an IL-15 variant comprising a D61Q substitution compared to SEQ ID No: 1; an IL-15 variant comprising a N65Q substitution compared to SEQ ID No: 1; an IL-15 variant comprising a I68A substitution compared to SEQ ID No: 1; an IL-15 variant comprising a L69A substitution compared to SEQ ID No: 1; an IL-15 variant comprising a L69V substitution compared to SEQ ID No: 1; an IL-15 variant comprising a Q108N substitution compared to SEQ ID No: 1; an IL-15 variant comprising S7N/K10Q/I68A substitutions compared to SEQ ID No: 1; an IL-15 variant comprising K10Q/I68A/L69V substitutions compared to SEQ ID No: 1; an IL-15 variant comprising S7N/K10Q/L69V substitutions compared to SEQ ID No: 1; an IL-15 variant comprising S7N/I68A/L69V substitutions compared to SEQ ID No: 1; an IL-15 variant comprising S7N/K10Q substitutions compared to SEQ ID No: 1; an IL-15 variant comprising S7N/I68A substitutions compared to SEQ ID No: 1; an IL-15 variant comprising S7N/L69V substitutions compared to SEQ ID No: 1; an IL-15 variant comprising K10Q/I68A substitutions compared to SEQ ID No: 1; an IL-15 variant comprising K10Q/L69V substitutions compared to SEQ ID No: 1; an IL-15 variant comprising I68A/L69V substitutions compared to SEQ ID No: 1; an IL-15 variant comprising a I68F substitution compared to SEQ ID No: 1; an IL-15 variant comprising a I68G substitution compared to SEQ ID No: 1; an IL-15 variant comprising a I68K substitution compared to SEQ ID No: 1; and an IL-15 variant comprising a I68R substitution compared to SEQ ID No: 1.

In some further embodiments, the IgG1 Fc domain as described above may comprise SEQ ID No: 3 in one chain and SEQ ID No: 4 in the other chain (referred to as “V47” format); alternatively, the IgG1 Fc domain as described above may comprise SEQ ID No: 5 in one chain and SEQ ID No: 6 in the other chain (referred to as “V42” format). For example, in certain embodiments, the IL-15 variant is operably linked to one chain of the Fc domain as set forth in SEQ ID No: 3 while the IL-15Rα protein domain is operably linked to the other chain of the Fc domain as set forth in SEQ ID No: 4; in some other embodiments, the IL-15 variant is operably linked to one chain of the Fc domain as set forth in SEQ ID No: 4 while the IL-15Rα protein domain is operably linked to the other chain of the Fc domain as set forth in SEQ ID No: 3; in certain embodiments, the IL-15 variant is operably linked to one chain of the Fe domain as set forth in SEQ ID No: 5 while the IL-15Rα protein domain is operably linked to the other chain of the Fe domain as set forth in SEQ ID No: 6; in some other embodiments, the IL-15 variant is operably linked to one chain of the Fe domain as set forth in SEQ ID No: 6 while the IL-15Rα protein domain is operably linked to the other chain of the Fe domain as set forth in SEQ ID No: 5.

In some exemplary embodiments, the IL-15/IL-15Rα-Fc fusion proteins as disclosed herein comprise two chains, the first chain comprises an IL-15 variant comprising a D8E substitution operably linked to an IgG1 Fc domain (V47 format), the second chain comprises a wild-type IL-15Rα protein operably linked to the other chain of the IgG1 Fc domain (V47 format). Alternatively, the Fe domain may be in V42 format.

In some exemplary embodiments, the IL-15/IL-15Rα-Fc fusion proteins as disclosed herein comprise two chains, the first chain comprises an IL-15 variant comprising a D8S substitution operably linked to an IgG1 Fc domain (V47), the second chain comprises a wild-type IL-15Rα protein operably linked to the other chain of the IgG1 Fc domain (V47). Alternatively, the Fe domain may be in V42 format.

In some exemplary embodiments, the IL-15/IL-15Rα-Fc fusion proteins as disclosed herein comprise two chains, the first chain comprises an IL-15 variant comprising a N65Q substitution operably linked to an IgG1 Fc domain (V47), the second chain comprises a wild-type IL-15Rα protein operably linked to the other chain of the IgG1 Fc domain (V47). Alternatively, the Fe domain may be in V42 format.

In some exemplary embodiments, the IL-15-Fc fusion proteins as disclosed herein comprise two chains, the first chain comprises an IL-15 variant comprising a L69A substitution operably linked to an IgG1 Fc domain (V47), the second chain comprises a wild-type IL-15Rα protein operably linked to the other chain of the IgG1 Fc domain (V47). Alternatively, the Fe domain may be in V42 format.

In some exemplary embodiments, the IL-15-Fc fusion proteins as disclosed herein comprise two chains, the first chain comprises an IL-15 variant comprising a Q108N substitution operably linked to an IgG1 Fc domain (V47), the second chain comprises a wild-type IL-15Rα protein operably linked to the other chain of the IgG1 Fc domain (V47). Alternatively, the Fe domain may be in V42 format.

In some exemplary embodiments, the IL-15-Fc fusion proteins as disclosed herein comprise two chains, the first chain comprises an IL-15 variant comprising a S7N+K10Q+I68A substitution operably linked to an IgG1 Fc domain (V47), the second chain comprises a wild-type IL-15Rα protein operably linked to the other chain of the IgG1 Fc domain (V47). Alternatively, the Fe domain may be in V42 format.

In some exemplary embodiments, the IL-15-Fc fusion proteins as disclosed herein comprise two chains, the first chain comprises an IL-15 variant comprising S7N+I68A+L69V substitutions operably linked to an IgG1 Fc domain (V47), the second chain comprises a wild-type IL-15Rα protein operably linked to the other chain of the IgG1 Fc domain (V47). Alternatively, the Fc domain may be in V42 format.

In some exemplary embodiments, the IL-15-Fc fusion proteins as disclosed herein comprise two chains, the first chain comprises an IL-15 variant comprising S7N+I68A substitutions operably linked to an IgG1 Fc domain (V47), the second chain comprises a wild-type IL-15Rα protein operably linked to the other chain of the IgG1 Fc domain (V47). Alternatively, the Fc domain may be in V42 format.

In some exemplary embodiments, the IL-15-Fc fusion proteins as disclosed herein comprise two chains, the first chain comprises an IL-15 variant comprising I68A+L69V substitutions operably linked to an IgG1 Fc domain (V47), the second chain comprises a wild-type IL-15Rα protein operably linked to the other chain of the IgG1 Fc domain (V47). Alternatively, the Fc domain may be in V42 format.

In some specific embodiments, the IL-15-Fc fusion proteins as disclosed herein comprise two chains, the first chain comprises an amino acid sequence as set forth in SEQ ID No: 7, 9, 11, 13, 15, 17, 19, 21 or 23, and/or the second chain comprises an amino acid sequence as set forth in SEQ ID No: 8, 10, 12, 14, 16, 18, 20 or 24.

In some exemplary embodiments, the IL-15-Fc fusion proteins as disclosed herein comprise two chains, the first chain comprises an IL-15 variant comprising an insertion between S7 and D8 operably linked to an IgG1 Fc domain (V805), the second chain comprises a wild-type IL-15Rα protein operably linked to the other chain of the IgG1 Fc domain (V805).

In some exemplary embodiments, the IL-15-Fc fusion proteins as disclosed herein comprise two chains, the first chain comprises an IL-15 variant comprising an insertion between D8 and L9 operably linked to an IgG1 Fc domain (V805, which is identical to V47 Fc format), the second chain comprises a wild-type IL-15Rα protein operably linked to the other chain of the IgG1 Fc domain (V805).

In some exemplary embodiments, the IL-15-Fc fusion proteins as disclosed herein comprise two chains, the first chain comprises an IL-15 variant comprising an insertion between L9 and K10 operably linked to an IgG1 Fc domain (V805), the second chain comprises a wild-type IL-15Rα protein operably linked to the other chain of the IgG1 Fc domain (V805).

In some exemplary embodiments, the IL-15-Fc fusion proteins as disclosed herein comprise two chains, the first chain comprises an IL-15 variant comprising an insertion between K10 and K11 operably linked to an IgG1 Fc domain (V805), the second chain comprises a wild-type IL-15Rα protein operably linked to the other chain of the IgG1 Fc domain (V805).

In some exemplary embodiments, the IL-15-Fc fusion proteins as disclosed herein comprise two chains, the first chain comprises an IL-15 variant comprising an insertion between E64 and N65 operably linked to an IgG1 Fc domain (V805), the second chain comprises a wild-type IL-15Rα protein operably linked to the other chain of the IgG1 Fc domain (V805).

In some exemplary embodiments, the IL-15-Fc fusion proteins as disclosed herein comprise two chains, the first chain comprises an IL-15 variant comprising an insertion between N65 and L66 operably linked to an IgG1 Fc domain (V805), the second chain comprises a wild-type IL-15Rα protein operably linked to the other chain of the IgG1 Fc domain (V805).

In some specific embodiments, the IL-15-Fc fusion proteins as disclosed herein comprise two chains, the first chain comprises an amino acid sequence as set forth in SEQ ID No: 25, 27, 29, 31, 33, or 35, and/or the second chain comprises an amino acid sequence as set forth in SEQ ID No: 26, 28, 30, 32, 34, or 36.

Properties of the IL-15 Variants and IL-15-Fc Fusion Proteins as Disclosed Herein

The present disclosure provides potency reduced, PK/PD enhanced IL-15 variants and IL-15-Fc fusion proteins comprising these IL-15 variants. The IL-15 variants have shown reduced potency/toxicity, prolonged PK, enhanced PD, and resulted in sustained lymphoexpansion in cyno monkeys, thus may serve as a novel immunotherapy agent with improved anti-tumor efficacy.

The functionality of these IL-15 variants and IL-15-Fc fusion proteins may be assessed in a number of ways, such as in vitro or in vivo assays.

In some embodiments, the effect of the IL-15 variants and IL-15-Fc fusion proteins is evaluated by immune cell proliferation assays, using for example Ki-67 intracellular staining of immune effector cells. Ki67 is a protein strictly associated with cell proliferation and the percentage of Ki67 on CD8+ T and NK cells may be measured by FACS, which indicates their activities in stimulating CD8+T and NK cells. Other immune cells can also be used for evaluating the effect on cell proliferation, such as CD45+ lymphocytes, among others.

In some embodiments, the effect of the IL-15 variants and IL-15-Fc fusion proteins is evaluated by quantifying a signaling pathway measured by phosphorylation of certain factors, such as STAT5 phosphorylation.

In some embodiments, the effect of the IL-15 variants and IL-15-Fc fusion proteins is evaluated by assessing T cell activity measured by cytokine production. As shown in the Examples, no obvious change in inflammatory cytokine production (e.g. IL-2, TNF, IFN-γ (Th1) and IL-4 (Th2)) was observed after the treatment of the IL-15 variants as disclosed herein.

In some embodiments, the effect of the IL-15 variants and IL-15-Fc fusion proteins is evaluated by assessing lympho-expansion (counts in WBCs, lymphocytes, monocytes and basophile).

The IL-15 comprising fusion proteins of the present disclosure provide at least one of the following properties:
(a) more moderate potency in simulating immune cell proliferation, such as CD8+ T cell and NK cell proliferation;
(b) prolonged pharmacokinetics profile without significant ADA production;
(c) shows a delayed yet higher lymphocyte proliferation in pharmacodynamics study;
(d) no distinct increase in inflammatory cytokine production; or
(e) no obvious toxicity in vivo.

Nucleic Acid Molecules Encoding the IL-1S Variants and Fusion Proteins

In some aspects, the disclosure is directed to an isolated nucleic acid molecule, comprising a nucleic acid sequence encoding the IL-15 variant or the heterodimeric Fc fusion protein as disclosed herein.

Nucleic acids of the disclosure can be obtained using standard molecular biology techniques. The isolated nucleic acid encoding the IL-15 variant can be operatively linked to another DNA molecule encoding an Fc domain. Similarly, a nucleic acid encoding the IL-15Rα domain (wild-type, variant or any fragment thereof that retains IL15 binding activity) can be operatively linked to another DNA molecule encoding an Fc domain. DNA fragments encompassing these regions can be obtained by standard PCR amplification.

Once DNA fragments encoding IL-15, IL-15Rα and Fc domains are obtained, these DNA fragments can be further manipulated by standard recombinant DNA techniques, for example incorporated into expression vectors as is known in the art. In some embodiments, nucleic acids encoding these DNA fragments are each contained within a single expression vector, generally under different or the same promoter control. In some other embodiments, nucleic acids encoding these DNA fragments are operably linked and contained in a single expression vector under the control of the same promoter. The term “operatively linked”, as used in this context, is intended to mean that the two DNA fragments are joined such that the amino acid sequences encoded by the two DNA fragments remain in-frame.

Vectors and Host Cells

The DNAs encoding the IL-15 variant or IL-15 comprising fusion proteins may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of desired proteins in the recombinant host cells. See, e.g., PCT Publication No. WO 87/04462.

Generally, a nucleic acid sequence encoding one or all chains of the fusion protein can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter. Regulatable promoters that include a repressor with the operon can be used.

Host cells as disclosed in the present disclosure may be any cell which is suitable for expressing the fusion proteins of the present disclosure, for instance, bacterial cells, yeast, mammalian cells. Mammalian host cells for expressing the fusion proteins of the present disclosure include Chinese Hamster Ovary (CHO cells) (including dhfr CHO cells, described in Urlaub and Chasin, (1980) Proc. Natl. Acad. Sci USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells.

In particular, for use with NSO myeloma cells, another expression system is the GS gene expression system disclosed in WO 87/04462, WO 89/01036 and EP 338,841. When recombinant expression vectors encoding the antibody are introduced into mammalian host cells, the fusion proteins are produced by culturing the host cells for a period of time sufficient to allow for expression of the fusion protein in the host cells or, secretion of the fusion protein into the culture medium in which the host cells are grown. The fusion proteins can be recovered from the culture medium using standard protein purification methods.

Pharmaceutical Compositions

In some aspects, the disclosure is directed to a pharmaceutical composition comprising the heterodimeric fusion protein as disclosed herein and a pharmaceutically acceptable carrier.

Components of the Compositions

The pharmaceutical composition may optionally contain one or more additional pharmaceutically active ingredients, such as an antibody. The pharmaceutical compositions of the disclosure also can be administered in a combination therapy with, for example, another immune-stimulatory agent, anti-cancer agent, an antiviral agent, or a vaccine. A pharmaceutically acceptable carrier can include, for example, a pharmaceutically acceptable liquid, gel or solid carriers, an aqueous medium, a non-aqueous medium, an anti-microbial agent, isotonic agents, buffers, antioxidants, anesthetics, suspending/dispersing agent, a chelating agent, a diluent, adjuvant, excipient or a nontoxic auxiliary substance, other known in the art various combinations of components or more.

Suitable components may include, for example, antioxidants, fillers, binders, disintegrating agents, buffers, preservatives, lubricants, flavorings, thickening agents, coloring agents, emulsifiers or stabilizers such as sugars and cyclodextrin. Suitable anti-oxidants may include, for example, methionine, ascorbic acid, EDTA, sodium thiosulfate, platinum, catalase, citric acid, cysteine, mercapto glycerol, thioglycolic acid, Mercapto sorbitol, butyl methyl anisole, butylated hydroxy toluene and/or propylgalacte. As disclosed in the present disclosure, the composition may include one or more anti-oxidants such as methionine, reducing antibody or antigen binding fragment thereof that may be oxidized. The oxidation reduction may prevent or reduce a decrease in binding affinity, thereby enhancing protein stability and extended shelf life. Thus, in some embodiments, the present disclosure provides a composition comprising fusion proteins and one or more anti-oxidants such as methionine. The present disclosure further provides a variety of methods, wherein a fusion protein is mixed with one or more anti-oxidants, such as methionine, so that the fusion protein can be prevented from oxidation, to extend their shelf life and/or increased activity.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

Administration, Formulation and Dosage

The pharmaceutical composition of the disclosure may be administered in vivo, to a subject in need thereof, by various routes, including, but not limited to, oral, intravenous, intra-arterial, subcutaneous, parenteral, intranasal, intramuscular, intracranial, intracardiac, intraventricular, intratracheal, buccal, rectal, intraperitoneal, intradermal, topical, transdermal, and intrathecal, or otherwise by implantation or inhalation. The subject compositions may be formulated into preparations in solid, semi-solid, liquid, or gaseous forms; including, but not limited to, tablets, capsules, powders, granules, ointments, solutions, suppositories, enemas, injections, inhalants, and aerosols. The appropriate formulation and route of administration may be selected according to the intended application and therapeutic regimen.

Suitable formulations for enteral administration include hard or soft gelatin capsules, pills, tablets, including coated tablets, elixirs, suspensions, syrups or inhalations and controlled release forms thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilizers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Similarly, the particular dosage regimen, including dose, timing and repetition, will depend on the particular individual and that individual's medical history, as well as empirical considerations such as pharmacokinetics (e.g., half-life, clearance rate, etc.).

Frequency of administration may be determined and adjusted over the course of therapy, and is based on reducing the number of proliferative or tumorigenic cells, maintaining the reduction of such neoplastic cells, reducing the proliferation of neoplastic cells, or delaying the development of metastasis. In some embodiments, the dosage administered may be adjusted or attenuated to manage potential side effects and/or toxicity. Alternatively, sustained continuous release formulations of a subject therapeutic composition may be appropriate.

It will be appreciated by one of skill in the art that appropriate dosages can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action that achieve the desired effect without causing substantial harmful or deleterious side-effects.

In general, the IL-15/IL-15Rα-Fc fusion proteins of the disclosure may be administered in various ranges. These include about 100 μg/kg body weight to about 10 mg/kg body weight per dose; about 100 μg/kg body weight to about 1 mg/kg body weight per dose; about 1 μg/kg body weight to about 10 mg/kg body weight per dose. Other ranges include about 100 μg/kg body weight to about 200 μg/kg body weight per dose; about 200 μg/kg body weight to about 300 μg/kg body weight per dose; about 300 μg/kg body weight to about 400 μg/kg body weight per dose; about 400 μg/kg body weight to about 0.5 μg/kg body weight per dose; and about 0.5 mg/kg body weight to about 1 mg/kg body weight per dose. In certain embodiments, the dosage is at least about 100 μg/kg body weight, at least about 250 μg/kg body weight, at least about 750 μg/kg body weight, at least about 3 mg/kg body weight, at least about 5 mg/kg body weight, at least about 10 mg/kg body weight.

In any event, the IL-15/IL-15Rα-Fc fusion proteins of the disclosure is preferably administered as needed to subjects in need thereof. Determination of the frequency of administration may be made by persons skilled in the art, such as an attending physician based on considerations of the condition being treated, age of the subject being treated, severity of the condition being treated, general state of health of the subject being treated and the like.

In certain preferred embodiments, the course of treatment involving the IL-15/IL-15Rα-Fe fusion proteins of the present disclosure will comprise multiple doses of the selected drug product over a period of weeks or months. More specifically, the IL-15/IL-15Rα-Fc fusion proteins of the present disclosure may be administered once every four days, every week, every ten days, every two weeks, every three weeks, every month, every six weeks, every two months, every ten weeks or every three months. In this regard, it will be appreciated that the dosages may be altered or the interval may be adjusted based on patient response and clinical practices.

Dosages and regimens may also be determined empirically for the disclosed therapeutic compositions in individuals who have been given one or more administration(s). For example, individuals may be given incremental dosages of a therapeutic composition produced as described herein. In selected embodiments, the dosage may be gradually increased or reduced or attenuated based respectively on empirically determined or observed side effects or toxicity. To assess efficacy of the selected composition, a marker of the specific disease, disorder or condition can be followed as described previously. For cancer, these include direct measurements of tumor size via palpation or visual observation, indirect measurement of tumor size by x-ray or other imaging techniques; an improvement as assessed by direct tumor biopsy and microscopic examination of the tumor sample; the measurement of an indirect tumor marker (e.g., PSA for prostate cancer) or a tumorigenic antigen identified according to the methods described herein, a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of neoplastic condition, the stage of neoplastic condition, whether the neoplastic condition has begun to metastasize to other location in the individual, and the past and concurrent treatments being used.

Compatible formulations for parenteral administration (e.g., intravenous injection) will comprise the IL-15/IL-15Rα-Fc fusion proteins as disclosed herein in concentrations that are considered suitable for administration and can be empirically determined by those skilled in the art, for example from about 10 μg/ml to about 10 mg/ml.

### APPLICATIONS OF THE DISCLOSURE

The IL-15/IL-15Rα-Fc fusion proteins, pharmaceutical compositions and methods of the present disclosure have numerous in vitro and in vivo utilities involving, for example, enhancement of immune response. For example, these molecules can be administered to cells in culture, in vitro or ex vivo, or to human subjects, e.g., in vivo, to enhance immunity in a variety of situations. The immune response can be modulated, for instance, augmented, stimulated or up-regulated.

For instance, the subjects include human patients in need of enhancement of an immune response. The methods are particularly suitable for treating human patients having a disorder that can be treated by augmenting an immune response (e.g., the NK/T-cell mediated immune response). In a particular embodiment, the methods are particularly suitable for treatment of cancer in vivo. To achieve enhancement of immunity, the IL-15/IL-15Rα-Fc fusion proteins can be administered alone or in combination with another therapy. When IL-15/IL-15Rα-Fc fusion proteins are administered together with another agent, the two can be administered in either order or simultaneously.

Treatment of Disorders Including Cancers and Infectious Diseases

In some aspects, the present disclosure provides a method of treating a disorder or a disease in a mammal, which comprises administering to the subject (for example, a human) in need of treatment a therapeutically effective amount of the IL-15/IL-15Rα-Fc fusion proteins as disclosed herein. The disorder or disease may be a cancer.

A variety of cancers, whether malignant or benign and whether primary or secondary, may be treated or prevented with a method provided by the disclosure. The cancers may be solid cancers or hematologic malignancies. Examples of such cancers include lung cancers such as bronchogenic carcinoma (e.g., non-small cell lung cancer, squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as colon cancer, leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblasts, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas. In a specific embodiment, the cancer is colon cancer.

In some embodiments, examples of cancer include but not limited to B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone-MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In some embodiments, examples of cancer further include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

In some embodiments, the IL-15 comprising fusion proteins as disclosed herein may be used to treat or prevent an infectious disease, which may be caused by a viral, bacterial, fungal or parasite infection. In some other embodiments, the IL-15 comprising fusion proteins as disclosed herein may be used to treat or prevent immunodeficiency or lymphopenia.

Stimulation of an Immune Response without Incurring Cytotoxicity

In some aspects, the disclosure also provides a method of enhancing (for example, stimulating) an immune response in a subject comprising administering an IL-15/IL-15Rα-Fc fusion protein of the disclosure to the subject such that an immune response in the subject is enhanced while no undesired side effects are presented. For example, the subject is a mammal. In a specific embodiment, the subject is a human.

The term “enhancing an immune response” or its grammatical variations, means stimulating, evoking, increasing, improving, or augmenting any response of a mammal's immune system. The immune response may be a cellular response (i.e. cell-mediated, such as cytotoxic T lymphocyte mediated) or a humoral response (i.e. antibody mediated response), and may be a primary or secondary immune response. Examples of enhancement of immune response include increased CD4⁺ helper T cell activity and generation of cytolytic T cells. The enhancement of immune response can be assessed using a number of in vitro or in vivo measurements known to those skilled in the art, including, but not limited to, cytotoxic T lymphocyte assays, release of cytokines (for example IL-2 production or IFN-γ production), regression of tumors, survival of tumor bearing animals, antibody production, immune cell proliferation, expression of cell surface markers, and cytotoxicity. Typically, methods of the disclosure enhance the immune response by a mammal when compared to the immune response by an untreated mammal or a mammal not treated using the methods as disclosed herein. In one embodiment, the immune response is cytokine production, particularly IFN-γ production or IL-12 production. In another embodiment, the immune response is enhanced B cell proliferation.

The IL-15 fusion proteins as disclosed herein may be used alone as a monotherapy, or more often, used in combination with cell immunotherapies, targeted therapies, chemical therapies or radiotherapies.

Combined Use with Cellular Immunotherapies

In some embodiments, the IL-15 fusion proteins as disclosed herein are used in combination with a cellular immunotherapy, also known as adoptive cell therapy. As is generally known, cellular immunotherapy is a form of treatment that uses the cells of human body's immune system to eliminate cancer. Some of these approaches involve directly isolating our own immune cells and simply expanding their numbers (e.g. performed by activating and expanding the immune cells of patient outside of the body and infused into the patient), whereas others involve genetically engineering immune cells (via gene therapy) to enhance their cancer-fighting capabilities. Cellular immunotherapies can be deployed in different ways, including but not limited to Tumor-Infiltrating Lymphocyte (TIL) therapy, Engineered T Cell Receptor (TCR-T) therapy, Chimeric Antigen Receptor (CAR) T Cell therapy, CAR NK Cell therapy, Natural Killer (NK) Cell therapy.

Combined Use with Targeted Therapies and Chemotherapies

The heterodimeric fusion proteins as disclosed herein may be administered as the sole active ingredient or in conjunction with, e.g. as an adjuvant to or in combination to, other drugs e.g. anti-cancer agents, immunomodulating agents or other anti-inflammatory agents, e.g. for the treatment or prevention of diseases mentioned above.

The term “anti-cancer agent” or “anti-proliferative agent” means any agent that can be used to treat a cell proliferative disorder such as cancer, and includes, but is not limited to, therapeutic antibodies, cytotoxic agents, cytostatic agents, anti-angiogenic agents, debulking agents, chemotherapeutic agents, radiotherapy and radiotherapeutic agents, targeted anti-cancer agents, BRMs, cancer vaccines, cytokines, hormone therapies, radiation therapy and anti-metastatic agents and immunotherapeutic agents.

For example, the heterodimeric fusion proteins as described herein may be used in combination with a wide variety of monoclonal antibodies, such as antibodies against tumor related antigens or pathways, e.g. PD-1/PD-L1, TIM-3, LAG-3, VEGF, HER2, CTLA-4; antibodies against leukocyte receptors, e.g., MHC, CD2, CD3, CD4, CD7, CD8, CD25, CD28, CD40, CD45, CD58, CD80, CD86 or their ligands; CD3 engager antibodies, NK engager antibodies; ADCC enabling anti-Tumor associated antigens; monoclonal antibodies to TNF, among others. The antibodies may include but not limited to, abciximab, adalimumab, alefacept, alemtuzumab, basiliximab, belimumab, bezlotoxumab, canakinumab, certolizumab pegol, cetuximab, daclizumab, denosumab, efalizumab, golimumab, inflectra, ipilimumab, ixekizumab, natalizumab, nivolumab, olaratumab, omalizumab, palivizumab, panitumumab, pembrolizumab, rituximab, tocilizumab, trastuzumab, secukinumab, and ustekinumab.

The antibodies may be monospecific or multi-specific. For example, the antibodies may be trispecific antibodies (TrAbs or TrioMabs), which have two variable segments for antigen binding and an Fc component to recruit immune cells. One example of TrAb is Catumaxomab for treating EpCam positive gastric and ovarian tumors. The antibodies may also be bispecific T cell engager antibodies (BiTE), such as Blinatumomab, MEHD7945A, ABT-122, XmAb5871 etc.

In some embodiments, the heterodimeric fusion proteins as described herein may be used in combination with immunomodulatory compounds, e.g. a recombinant binding molecule having at least a portion of the extracellular domain of CTLA4 or a mutant thereof, adhesion molecule inhibitors, e.g. LFA-1 antagonists, ICAM-1 or -3 antagonists, VCAM-4 antagonists or VLA-4 antagonists; blockers of proinflammatory cytokines, IL-1 blockers; chemokines blockers; or a chemotherapeutic agent.

For the purposes of the present disclosure a “chemotherapeutic agent” comprises a chemical compound that non-specifically decreases or inhibits the growth, proliferation, and/or survival of cancer cells (e.g., cytotoxic or cytostatic agents). Such chemical agents are often directed to intracellular processes necessary for cell growth or division, and are thus particularly effective against cancerous cells, which generally grow and divide rapidly. For example, vincristine depolymerizes microtubules, and thus inhibits cells from entering mitosis.

In general, chemotherapeutic agents can include any chemical agent that inhibits, or is designed to inhibit, a cancerous cell or a cell likely to become cancerous or generate tumorigenic progeny (e.g., TIC). Such agents are often administered, and are often most effective, in combination, e.g., in regimens such as CHOP or FOLFIRI. Examples of anti-cancer agents that may be used in combination with the site-specific constructs of the present disclosure (either as a component of a site specific conjugate or in an unconjugated state) include, but are not limited to, e.g. paclitaxel, gemcitabine, cisplatinum, doxorubicin, 5-fluorouracil, capecitabine, combretastatin, leucovorin etc.

For example, the heterodimeric fusion proteins as described herein may be used in combination with DMARD, e.g. Gold salts, sulphasalazine, antimalarias, methotrexate, D-penicillamine, azathioprine, mycophenolic acid, cyclosporine A, tacrolimus, sirolimus, minocycline, lefiunomide, glococorticoids; a calcineurin inhibitor, e.g. cyclosporin A or FK 506; a modulator of lymphocyte recirculation, e.g. FTY720 and FTY720 analogs; a mTOR inhibitor, e.g. rapamycin, 40-O-(2-hydroxyethyl)-rapamycin, CC1779, ABT578, AP23573 or TAFA-93; an ascomycin having immunosuppressive properties, e.g. ABT-281, ASM981, etc.; corticosteroids; cyclo-phosphamide; azathioprene; methotrexate; lefiunomide; mizoribine; mycophenolic acid; myco-phenolate mofetil; 15-deoxyspergualine or an immunosuppressive homologue, analogue or derivative thereof, immunosuppressive

It will be appreciated that, in selected embodiments as discussed above, such anti-cancer agents may comprise conjugates and may be associated with the disclosed IL-15/IL-15Rα-Fc fusion proteins prior to administration. More specifically, in certain embodiments selected anti-cancer agents will be linked to the unpaired cysteines of the engineered IL-15/IL-15Rα-Fc fusion proteins to provide engineered conjugates as set forth herein. Accordingly, such engineered conjugates are expressly contemplated as being within the scope of the present disclosure. In other embodiments, the disclosed anti-cancer agents will be given in combination with site-specific conjugates comprising a different therapeutic agent as set forth above.

It will be easily appreciated that, the anti-cancer agents or immunomodulating agents to be used in combination with the IL-15 fusion proteins as disclosed herein should be compatible with the IL-15 fusion proteins, i.e. would not reduce, disturb, or eliminate the effect of the IL-15 fusion proteins as disclosed herein, and preferably provide a coordinating or even synergistic effect.

As an Immune Enhancing Component/Moiety in a Multi-Specific Antibody

The IL-15 fusion protein and heterodimeric fusion proteins as disclosed herein may be associated with an antigen-specific binding moiety to form a multi-specific antibody complex. For example, an antigen-binding moiety (e.g. comprising a heavy chain variable region and a light chain variable region) may be fused with the N terminal or C terminal of the IL-15 protein domain. Such multi-specific antibody complex not only have a high affinity to a targeted antigen, but also the potency of IL-15 in promoting immune cell proliferation.

Combined Use with Radiotherapies

The present disclosure also provides for the combination of the IL-15/IL-15Rα-Fc fusion proteins thereof with radiotherapy (i.e., any mechanism for inducing DNA damage locally within tumor cells such as gamma-irradiation, X-rays, UV-irradiation, microwaves, electronic emissions and the like). Combination therapy using the directed delivery of radioisotopes to tumor cells is also contemplated, and the disclosed conjugates may be used in connection with a targeted anti-cancer agent or other targeting means. Typically, radiation therapy is administered in pulses over a period of time from about 1 to about 2 weeks. The radiation therapy may be administered to subjects having head and neck cancer for about 6 to 7 weeks. Optionally, the radiation therapy may be administered as a single dose or as multiple, sequential doses.

Pharmaceutical Packs and Kits

Pharmaceutical packs and kits comprising one or more containers, comprising one or more doses of the IL-15/IL-15Rα-Fc fusion proteins are also provided. In certain embodiments, a unit dosage is provided wherein the unit dosage contains a predetermined amount of a composition comprising, for example, the IL-15/IL-15Rα-Fc fusion proteins, with or without one or more additional agents. For other embodiments, such a unit dosage is supplied in single-use prefilled syringe for injection. In still other embodiments, the composition contained in the unit dosage may comprise saline, sucrose, or the like; a buffer, such as phosphate, or the like; and/or be formulated within a stable and effective pH range. Alternatively, in certain embodiments, the conjugate composition may be provided as a lyophilized powder that may be reconstituted upon addition of an appropriate liquid, for example, sterile water or saline solution. In certain preferred embodiments, the composition comprises one or more substances that inhibit protein aggregation, including, but not limited to, sucrose and arginine. Any label on, or associated with, the container(s) indicates that the enclosed conjugate composition is used for treating the neoplastic disease condition of choice.

The present disclosure also provides kits for producing single-dose or multi-dose administration units of site-specific conjugates and, optionally, one or more anti-cancer agents. The kit comprises a container and a label or package insert on or associated with the container. Suitable containers include, for example, bottles, vials, syringes, etc. The containers may be formed from a variety of materials such as glass or plastic and contain a pharmaceutically effective amount of the disclosed conjugates in a conjugated or unconjugated form. In other preferred embodiments, the container(s) comprise a sterile access port (for example the container may be an intravenous solution bag or a vial having a stopper pierceable by a hypodermic injection needle). Such kits will generally contain in a suitable container a pharmaceutically acceptable formulation of the engineered conjugate and, optionally, one or more anti-cancer agents in the same or different containers. The kits may also contain other pharmaceutically acceptable formulations, either for diagnosis or combined therapy. For example, in addition to the IL-15/IL-15Rα-Fc fusion proteins of the disclosure such kits may contain any one or more of a range of anti-cancer agents such as chemotherapeutic or radiotherapeutic drugs; anti-angiogenic agents; anti-metastatic agents; targeted anti-cancer agents; cytotoxic agents; and/or other anti-cancer agents.

More specifically the kits may have a single container that contains the disclosed the IL-15/IL-15Rα-Fc fusion proteins, with or without additional components, or they may have distinct containers for each desired agent. Where combined therapeutics are provided for conjugation, a single solution may be pre-mixed, either in a molar equivalent combination, or with one component in excess of the other. Alternatively, the conjugates and any optional anti-cancer agent of the kit may be maintained separately within distinct containers prior to administration to a patient. The kits may also comprise a second/third container means for containing a sterile, pharmaceutically acceptable buffer or other diluents such as bacteriostatic water for injection (BWFJ), phosphate-buffered saline (PBS), Ringer's solution and dextrose solution.

When the components of the kit are provided in one or more liquid solutions, the liquid solution is preferably an aqueous solution, with a sterile aqueous or saline solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

As indicated briefly above the kits may also contain a means by which to administer the IL-15/IL-15Rα-Fc fusion proteins and any optional components to a patient, e.g., one or more needles, I.V. bags or syringes, or even an eye dropper, pipette, or other such like apparatus, from which the formulation may be injected or introduced into the animal or applied to a diseased area of the body. The kits of the present disclosure will also typically include a means for containing the vials, or such like, and other component in close confinement for commercial sale, such as, e.g., injection or blow-molded plastic containers into which the desired vials and other apparatus are placed and retained.

Sequence Listing Summary

The following Table A provides a summary of the included sequences. The designation of the fusion proteins “W369-E17-TxU1-V00XX.uIgG1V42” or “W369-E17-TxU1-V00XX.uIgG1V47” or “W327151-TXU1.P1-V000X.uIgG1V805” may be abbreviated into the format as “V00XX.uIgG1V42”/“V00XX-V42” or “V00XX.uIgG1V47”/“V00XX-V47” or “V000X.uIgG1V805”/“V000X-V805”, respectively, throughout the specification.

TABLE A

| ID | Amino acid sequence/annotation |
|---|---|
| SEQ ID No: 1 | Human wild-type IL-15 protein |
| | NWVNVISDLKKIEDLIQSMHIDATLYTESDVHPSCKVTAMKCFLL |
| | ELQVISLESGDASIHDTVENLIILANNSLSSNGNVTESGCKECEEL |
| | EEKNIKEFLQSFVHIVQMFINTS |
| SEQ ID No: 2 | Human wild-type IL-15Rα protein fragment, IL-15 binding domain |
| | ITCPPPMSVEHADIWVKSYSLYSRERYICNSGFKRKAGTSSLTEC |
| | VLNKATNVAHWTTPSLKCIR |
| SEQ ID No: 3 | One chain of the IgG1 Fc variant (V47, same as V805) |
| | EPKSSDKTHTCPPCPAPELLRGPSVFLFPPKPKDTLYITREPEVTC |
| | VVVDVSHEDPEVKFNWYVDGVEVHNAKTKPREEQYNSTYRVV |
| | SVLTVLHQDWLNGKEYKCKVSNKARPAPIEKTISKAKGQPREPQ |
| | VYTLPPCREEMTKNQVSLWCLVKGFYPSDIAVEWESNGQPENN |
| | YKTTPPVLDSDGSFFLYSKLTVDKSRWQQGNVFSCSVMHEALH |
| | NHYTQKSLSLSPG |
| SEQ ID No: 4 | 2nd chain of the IgG1 Fc variant (V47, same as V805) |
| | EPKSSDKTHTCPPCPAPELLRGPSVFLFPPKPKDTLYITREPEVTC |
| | VVVDVSHEDPEVKFNWYVDGVEVHNAKTKPREEQYNSTYRVV |
| | SVLTVLHQDWLNGKEYKCKVSNKARPAPIEKTISKAKGQPREPQ |
| | VCTLPPSREEMTKNQVSLSCAVKGFYPSDIAVEWESNGQPENNY |
| | KTTPPVLDSDGSFFLVSKLTVDKSRWQQGNVFSCSVMHEALHN |
| | HYTQKSLSLSPG |
| SEQ ID No: 5 | 1st chain of the IgG1 Fc variant (V42) |
| | EPKSSDKTHTCPPCPAPELLRGPSVFLFPPKPKDTLMISRTPEVTC |
| | VVVDVSHEDPEVKFNWYVDGVEVHNAKTKPREEQYNSTYRVV |
| | SVLTVLHQDWLNGKEYKCKVSNKARPAPIEKTISKAKGQPREPQ |
| | VYTLPPCREEMTKNQVSLWCLVKGFYPSDIAVEWESNGQPENN |
| | YKTTPPVLDSDGSFFLYSKLTVDKSRWQQGNVFSCSVMHEALH |
| | NHYTQKSLSLSPG |
| SEQ ID No: 6 | 2nd chain of the IgG1 Fc variant (V42) |
| | EPKSSDKTHTCPPCPAPELLRGPSVFLFPPKPKDTLMISRTPEVTC |
| | VVVDVSHEDPEVKFNWYVDGVEVHNAKTKPREEQYNSTYRVV |
| | SVLTVLHQDWLNGKEYKCKVSNKARPAPIEKTISKAKGQPREPQ |
| | VCTLPPSREEMTKNQVSLSCAVKGFYPSDIAVEWESNGQPENNY |
| | KTTPPVLDSDGSFFLVSKLTVDKSRWQQGNVFSCSVMHEALHN |
| | HYTQKSLSLSPG |
| SEQ ID No: 7 | 1st chain of V0005.uIgG1V47 |
| | NWVNVISELKKIEDLIQSMHIDATLYTESDVHPSCKVTAMKCFLL |
| | ELQVISLESGDASIHDTVENLIILANNSLSSNGNVTESGCKECEEL |
| | EEKNIKEFLQSFVHIVQMFINTSGGGGSEPKSSDKTHTCPPCPAPE |
| | LLRGPSVFLFPPKPKDTLYITREPEVTCVVVDVSHEDPEVKFNWY |
| | VDGVEVHNAKTKPREEQYNSTYRVVSVLTVLHQDWLNGKEYK |
| | CKVSNKARPAPIEKTISKAKGQPREPQVYTLPPCREEMTKNQVSL |
| | WCLVKGFYPSDIAVEWESNGQPENNYKTTPPVLDSDGSFFLYSK |
| | LTVDKSRWQQGNVFSCSVMHEALHNHYTQKSLSLSPG |
| SEQ ID No: 8 | 2nd chain of V0005.uIgG1V47 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPELL |
| | RGPSVFLFPPKPKDTLYITREPEVTCVVVDVSHEDPEVKFNWYV |
| | DGVEVHNAKTKPREEQYNSTYRVVSVLTVLHQDWLNGKEYKC |
| | KVSNKARPAPIEKTISKAKGQPREPQVCTLPPSREEMTKNQVSLS |
| | CAVKGFYPSDIAVEWESNGQPENNYKTTPPVLDSDGSFFLVSKLT |
| | VDKSRWQQGNVFSCSVMHEALHNHYTQKSLSLSPG |
| SEQ ID No: 9 | 1st chain of V0006.uIgG1V47 |
| | NWVNVISSLKKIEDLIQSMHIDATLYTESDVHPSCKVTAMKCFLL |
| | ELQVISLESGDASIHDTVENLIILANNSLSSNGNVTESGCKECEEL |
| | EEKNIKEFLQSFVHIVQMFINTSGGGGSEPKSSDKTHTCPPCPAPE |
| | LLRGPSVFLFPPKPKDTLYITREPEVTCVVVDVSHEDPEVKFNWY |
| | VDGVEVHNAKTKPREEQYNSTYRVVSVLTVLHQDWLNGKEYK |
| | CKVSNKARPAPIEKTISKAKGQPREPQVYTLPPCREEMTKNQVSL |
| | WCLVKGFYPSDIAVEWESNGQPENNYKTTPPVLDSDGSFFLYSK |
| | LTVDKSRWQQGNVFSCSVMHEALHNHYTQKSLSLSPG |
| SEQ ID No: 10 | 2nd chain of V0006.uIgG1V47 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPELL |
| | RGPSVFLFPPKPKDTLYITREPEVTCVVVDVSHEDPEVKFNWYV |
| | DGVEVHNAKTKPREEQYNSTYRVVSVLTVLHQDWLNGKEYKC |
| | KVSNKARPAPIEKTISKAKGQPREPQVCTLPPSREEMTKNQVSLS |
| | CAVKGFYPSDIAVEWESNGQPENNYKTTPPVLDSDGSFFLVSKLT |
| | VDKSRWQQGNVFSCSVMHEALHNHYTQKSLSLSPG |
| SEQ ID No: 11 | 1st chain of V0015.uIgG1V47 |
| | NWVNVISDLKKIEDLIQSMHIDATLYTESDVHPSCKVTAMKCFLL |
| | ELQVISLESGDASIHDTVEQLIILANNSLSSNGNVTESGCKECEEL |
| | EEKNIKEFLQSFVHIVQMFINTSGGGGSEPKSSDKTHTCPPCPAPE |
| | LLRGPSVFLFPPKPKDTLYITREPEVTCVVVDVSHEDPEVKFNWY |
| | VDGVEVHNAKTKPREEQYNSTYRVVSVLTVLHQDWLNGKEYK |
| | CKVSNKARPAPIEKTISKAKGQPREPQVYTLPPCREEMTKNQVSL |
| | WCLVKGFYPSDIAVEWESNGQPENNYKTTPPVLDSDGSFFLYSK |
| | LTVDKSRWQQGNVFSCSVMHEALHNHYTQKSLSLSPG |
| SEQ ID No: 12 | 2nd chain of V0015.uIgG1V47 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPELL |
| | RGPSVFLFPPKPKDTLYITREPEVTCVVVDVSHEDPEVKFNWYV |
| | DGVEVHNAKTKPREEQYNSTYRVVSVLTVLHQDWLNGKEYKC |
| | KVSNKARPAPIEKTISKAKGQPREPQVCTLPPSREEMTKNQVSLS |
| | CAVKGFYPSDIAVEWESNGQPENNYKTTPPVLDSDGSFFLVSKLT |
| | VDKSRWQQGNVFSCSVMHEALHNHYTQKSLSLSPG |
| SEQ ID No: 13 | 1st chain of V0018.uIgG1V47 |
| | NWVNVISDLKKIEDLIQSMHIDATLYTESDVHPSCKVTAMKCFLL |
| | ELQVISLESGDASIHDTVENLIIAANNSLSSNGNVTESGCKECEEL |
| | EEKNIKEFLQSFVHIVQMFINTSGGGGSEPKSSDKTHTCPPCPAPE |
| | LLRGPSVFLFPPKPKDTLYITREPEVTCVVVDVSHEDPEVKFNWY |
| | VDGVEVHNAKTKPREEQYNSTYRVVSVLTVLHQDWLNGKEYK |
| | CKVSNKARPAPIEKTISKAKGQPREPQVYTLPPCREEMTKNQVSL |
| | WCLVKGFYPSDIAVEWESNGQPENNYKTTPPVLDSDGSFFLYSK |
| | LTVDKSRWQQGNVFSCSVMHEALHNHYTQKSLSLSPG |
| SEQ ID No: 14 | 2nd chain of V0018.uIgG1V47 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPELL |
| | RGPSVFLFPPKPKDTLYITREPEVTCVVVDVSHEDPEVKFNWYV |
| | DGVEVHNAKTKPREEQYNSTYRVVSVLTVLHQDWLNGKEYKC |
| | KVSNKARPAPIEKTISKAKGQPREPQVCTLPPSREEMTKNQVSLS |
| | CAVKGFYPSDIAVEWESNGQPENNYKTTPPVLDSDGSFFLVSKLT |
| | VDKSRWQQGNVFSCSVMHEALHNHYTQKSLSLSPG |
| SEQ ID No: 15 | 1st chain of V0020.uIgG1V47 |
| | NWVNVISDLKKIEDLIQSMHIDATLYTESDVHPSCKVTAMKCFLL |
| | ELQVISLESGDASIHDTVENLIILANNSLSSNGNVTESGCKECEEL |
| | EEKNIKEFLQSFVHIVNMFINTSGGGGSEPKSSDKTHTCPPCPAPE |
| | LLRGPSVFLFPPKPKDTLYITREPEVTCVVVDVSHEDPEVKFNWY |
| | VDGVEVHNAKTKPREEQYNSTYRVVSVLTVLHQDWLNGKEYK |
| | CKVSNKARPAPIEKTISKAKGQPREPQVYTLPPCREEMTKNQVSL |
| | WCLVKGFYPSDIAVEWESNGQPENNYKTTPPVLDSDGSFFLYSK |
| | LTVDKSRWQQGNVFSCSVMHEALHNHYTQKSLSLSPG |
| SEQ ID No: 16 | 2nd chain of V0020.uIgG1V47 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPELL |
| | RGPSVFLFPPKPKDTLYITREPEVTCVVVDVSHEDPEVKFNWYV |
| | DGVEVHNAKTKPREEQYNSTYRVVSVLTVLHQDWLNGKEYKC |
| | KVSNKARPAPIEKTISKAKGQPREPQVCTLPPSREEMTKNQVSLS |
| | CAVKGFYPSDIAVEWESNGQPENNYKTTPPVLDSDGSFFLVSKLT |
| | VDKSRWQQGNVFSCSVMHEALHNHYTQKSLSLSPG |
| SEQ ID No: 17 | 1st chain of V0058.uIgG1V47 |
| | NWVNVINDLQKIEDLIQSMHIDATLYTESDVHPSCKVTAMKCFL |
| | LELQVISLESGDASIHDTVENLIALANNSLSSNGNVTESGCKECEE |
| | LEEKNIKEFLQSFVHIVQMFINTSGGGGSEPKSSDKTHTCPPCPAP |
| | ELLRGPSVFLFPPKPKDTLYITREPEVTCVVVDVSHEDPEVKFNW |
| | YVDGVEVHNAKTKPREEQYNSTYRVVSVLTVLHQDWLNGKEY |
| | KCKVSNKARPAPIEKTISKAKGQPREPQVYTLPPCREEMTKNQVS |
| | LWCLVKGFYPSDIAVEWESNGQPENNYKTTPPVLDSDGSFFLYS |
| | KLTVDKSRWQQGNVFSCSVMHEALHNHYTQKSLSLSPG |
| SEQ ID No: 18 | 2nd chain of V0058.uIgG1V47 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPELL |
| | RGPSVFLFPPKPKDTLYITREPEVTCVVVDVSHEDPEVKFNWYV |
| | DGVEVHNAKTKPREEQYNSTYRVVSVLTVLHQDWLNGKEYKC |
| | KVSNKARPAPIEKTISKAKGQPREPQVCTLPPSREEMTKNQVSLS |
| | CAVKGFYPSDIAVEWESNGQPENNYKTTPPVLDSDGSFFLVSKLT |
| | VDKSRWQQGNVFSCSVMHEALHNHYTQKSLSLSPG |
| SEQ ID No: 19 | 1st chain of V0061.uIgG1V47 |
| | NWVNVINDLKKIEDLIQSMHIDATLYTESDVHPSCKVTAMKCFL |
| | LELQVISLESGDASIHDTVENLIAVANNSLSSNGNVTESGCKECEE |
| | LEEKNIKEFLQSFVHIVQMFINTSGGGGSEPKSSDKTHTCPPCPAP |
| | ELLRGPSVFLFPPKPKDTLYITREPEVTCVVVDVSHEDPEVKFNW |
| | YVDGVEVHNAKTKPREEQYNSTYRVVSVLTVLHQDWLNGKEY |
| | KCKVSNKARPAPIEKTISKAKGQPREPQVYTLPPCREEMTKNQVS |
| | LWCLVKGFYPSDIAVEWESNGQPENNYKTTPPVLDSDGSFFLYS |
| | KLTVDKSRWQQGNVFSCSVMHEALHNHYTQKSLSLSPG |
| SEQ ID No: 20 | 2nd chain of V0061.uIgG1V47 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPELL |
| | RGPSVFLFPPKPKDTLYITREPEVTCVVVDVSHEDPEVKFNWYV |
| | DGVEVHNAKTKPREEQYNSTYRVVSVLTVLHQDWLNGKEYKC |
| | KVSNKARPAPIEKTISKAKGQPREPQVCTLPPSREEMTKNQVSLS |
| | CAVKGFYPSDIA VEWESNGQPENNYKTTPPVLDSDGSFFLVSKLT |
| | VDKSRWQQGNVFSCSVMHEALHNHYTQKSLSLSPG |
| SEQ ID No: 21 | 1st chain of V0063.uIgG1V47 |
| | NWVNVINDLKKIEDLIQSMHIDATLYTESDVHPSCKVTAMKCFL |
| | LELQVISLESGDASIHDTVENLIALANNSLSSNGNVTESGCKECEE |
| | LEEKNIKEFLQSFVHIVQMFINTSGGGGSEPKSSDKTHTCPPCPAP |
| | ELLRGPSVFLFPPKPKDTLYITREPEVTCVVVDVSHEDPEVKFNW |
| | YVDGVEVHNAKTKPREEQYNSTYRVVSVLTVLHQDWLNGKEY |
| | KCKVSNKARPAPIEKTISKAKGQPREPQVYTLPPCREEMTKNQVS |
| | LWCLVKGFYPSDIAVEWESNGQPENNYKTTPPVLDSDGSFFLYS |
| | KLTVDKSRWQQGNVFSCSVMHEALHNHYTQKSLSLSPG |
| SEQ ID No: 22 | 2nd chain of V0063.uIgG1V47 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPELL |
| | RGPSVFLFPPKPKDTLYITREPEVTCVVVDVSHEDPEVKFNWYV |
| | DGVEVHNAKTKPREEQYNSTYRVVSVLTVLHQDWLNGKEYKC |
| | KVSNKARPAPIEKTISKAKGQPREPQVCTLPPSREEMTKNQVSLS |
| | CAVKGFYPSDIAVEWESNGQPENNYKTTPPVLDSDGSFFLVSKLT |
| | VDKSRWQQGNVFSCSVMHEALHNHYTQKSLSLSPG |
| SEQ ID No: 23 | 1st chain of V0067.uIgG1V47 |
| | NWVNVISDLKKIEDLIQSMHIDATLYTESDVHPSCKVTAMKCFLL |
| | ELQVISLESGDASIHDTVENLIAVANNSLSSNGNVTESGCKECEEL |
| | EEKNIKEFLQSFVHIVQMFINTSGGGGSEPKSSDKTHTCPPCPAPE |
| | LLRGPSVFLFPPKPKDTLYITREPEVTCVVVDVSHEDPEVKFNWY |
| | VDGVEVHNAKTKPREEQYNSTYRVVSVLTVLHQDWLNGKEYK |
| | CKVSNKARPAPIEKTISKAKGQPREPQVYTLPPCREEMTKNQVSL |
| | WCLVKGFYPSDIAVEWESNGQPENNYKTTPPVLDSDGSFFLYSK |
| | LTVDKSRWQQGNVFSCSVMHEALHNHYTQKSLSLSPG |
| SEQ ID No: 24 | 2nd chain of V0067.uIgG1V47 |
| | ITCPPPMSVEHADIWVKSYSLYSRERYICNSGFKRKAGTSSLTEC |
| | VLNKATNVAHWTTPSLKCIRGGGGSEPKSSDKTHTCPPCPAPELL |
| | RGPSVFLFPPKPKDTLYITREPEVTCVVVDVSHEDPEVKFNWYV |
| | DGVEVHNAKTKPREEQYNSTYRVVSVLTVLHQDWLNGKEYKC |
| | KVSNKARPAPIEKTISKAKGQPREPQVCTLPPSREEMTKNQVSLS |
| | CAVKGFYPSDIAVEWESNGQPENNYKTTPPVLDSDGSFFLVSKLT |
| | VDKSRWQQGNVFSCSVMHEALHNHYTQKSLSLSPG |
| SEQ ID No: 25 | 1st chain of V0001.uIgG1V805 |
| | NWVNVISADLKKIEDLIQSMHIDATLYTESDVHPSCKVTAMKCF |
| | LLELQVISLESGDASIHDTVENLIILANNSLSSNGNVTESGCKECE |
| | ELEEKNIKEFLQSFVHIVQMFINTS |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVYTLPPCREEMTKNQVSLWCLVKGFYPSDIAVEWESNGQP |
| | ENNYKTTPPVLDSDGSFFLYSKLTVDKSRWQQGNVFSCSVMHE |
| | ALHNHYTQKSLSLSPG |
| SEQ ID No: 26 | 2nd chain of V0001.uIgG1V805 |
| | ITCPPPMSVEHADIWVKSYSLYSRERYICNSGFKRKAGTSSLTEC |
| | VLNKATNVAHWTTPSLKCIR |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVCTLPPSREEMTKNQVSLSCAVKGFYPSDIAVEWESNGQP |
| | ENNYKTTPPVLDSDGSFFLVSKLTVDKSRWQQGNVFSCSVMHE |
| | ALHNHYTQKSLSLSPG |
| SEQ ID No: 27 | 1st chain of V0002.uIgG1V805 |
| | NWVNVISDALKKIEDLIQSMHIDATLYTESDVHPSCKVTAMKCF |
| | LLELQVISLESGDASIHDTVENLIILANNSLSSNGNVTESGCKECE |
| | ELEEKNIKEFLQSFVHIVQMFINTS |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVYTLPPCREEMTKNQVSLWCLVKGFYPSDIAVEWESNGQP |
| | ENNYKTTPPVLDSDGSFFLYSKLTVDKSRWQQGNVFSCSVMHE |
| | ALHNHYTQKSLSLSPG |
| SEQ ID No: 28 | 2nd chain of V0002.uIgG1V805 |
| | ITCPPPMSVEHADIWVKSYSLYSRERYICNSGFKRKAGTSSLTEC |
| | VLNKATNVAHWTTPSLKCIR |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVCTLPPSREEMTKNQVSLSCAVKGFYPSDIAVEWESNGQP |
| | ENNYKTTPPVLDSDGSFFLVSKLTVDKSRWQQGNVFSCSVMHE |
| | ALHNHYTQKSLSLSPG |
| SEQ ID No: 29 | 1st chain of V0003.uIgG1V805 |
| | NWVNVISDLAKKIEDLIQSMHIDATLYTESDVHPSCKVTAMKCF |
| | LLELQVISLESGDASIHDTVENLIILANNSLSSNGNVTESGCKECE |
| | ELEEKNIKEFLQSFVHIVQMFINTS |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVYTLPPCREEMTKNQVSLWCLVKGFYPSDIAVEWESNGQP |
| | ENNYKTTPPVLDSDGSFFLYSKLTVDKSRWQQGNVFSCSVMHE |
| | ALHNHYTQKSLSLSPG |
| SEQ ID No: 30 | 2nd chain of V0003.uIgG1V805 |
| | ITCPPPMSVEHADIWVKSYSLYSRERYICNSGFKRKAGTSSLTEC |
| | VLNKATNVAHWTTPSLKCIR |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVCTLPPSREEMTKNQVSLSCAVKGFYPSDIAVEWESNGQP |
| | ENNYKTTPPVLDSDGSFFLVSKLTVDKSRWQQGNVFSCSVMHE |
| | ALHNHYTQKSLSLSPG |
| SEQ ID No: 31 | 1st chain of V0004.uIgG1V805 |
| | NWVNVISDLKAKIEDLIQSMHIDATLYTESDVHPSCKVTAMKCF |
| | LLELQVISLESGDASIHDTVENLIILANNSLSSNGNVTESGCKECE |
| | ELEEKNIKEFLQSFVHIVQMFINTS |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVYTLPPCREEMTKNQVSLWCLVKGFYPSDIAVEWESNGQP |
| | ENNYKTTPPVLDSDGSFFLYSKLTVDKSRWQQGNVFSCSVMHE |
| | ALHNHYTQKSLSLSPG |
| SEQ ID No: 32 | 2nd chain of V0004.uIgG1V805 |
| | ITCPPPMSVEHADIWVKSYSLYSRERYICNSGFKRKAGTSSLTEC |
| | VLNKATNVAHWTTPSLKCIR |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVCTLPPSREEMTKNQVSLSCAVKGFYPSDIAVEWESNGQP |
| | ENNYKTTPPVLDSDGSFFLVSKLTVDKSRWQQGNVFSCSVMHE |
| | ALHNHYTQKSLSLSPG |
| SEQ ID No: 33 | 1st chain of V0005.uIgG1V805 |
| | NWVNVISDLKKIEDLIQSMHIDATLYTESDVHPSCKVTAMKCFLL |
| | ELQVISLESGDASIHDTVEANLIILANNSLSSNGNVTESGCKECEE |
| | LEEKNIKEFLQSFVHIVQMFINTS |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVYTLPPCREEMTKNQVSLWCLVKGFYPSDIAVEWESNGQP |
| | ENNYKTTPPVLDSDGSFFLYSKLTVDKSRWQQGNVFSCSVMHE |
| | ALHNHYTQKSLSLSPG |
| SEQ ID No: 34 | 2nd chain of V0005.uIgG1V805 |
| | ITCPPPMSVEHADIWVKSYSLYSRERYICNSGFKRKAGTSSLTEC |
| | VLNKATNVAHWTTPSLKCIR |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVCTLPPSREEMTKNQVSLSCAVKGFYPSDIAVEWESNGQP |
| | ENNYKTTPPVLDSDGSFFLVSKLTVDKSRWQQGNVFSCSVMHE |
| | ALHNHYTQKSLSLSPG |
| SEQ ID No: 35 | 1st chain of V0006.uIgG1V805 |
| | NWVNVISDLKKIEDLIQSMHIDATLYTESDVHPSCKVTAMKCFLL |
| | ELQVISLESGDASIHDTVENALIILANNSLSSNGNVTESGCKECEE |
| | LEEKNIKEFLQSFVHIVQMFINTS |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVYTLPPCREEMTKNQVSLWCLVKGFYPSDIAVEWESNGQP |
| | ENNYKTTPPVLDSDGSFFLYSKLTVDKSRWQQGNVFSCSVMHE |
| | ALHNHYTQKSLSLSPG |
| SEQ ID No: 36 | 2nd chain of V0006.uIgG1V805 |
| | ITCPPPMSVEHADIWVKSYSLYSRERYICNSGFKRKAGTSSLTEC |
| | VLNKATNVAHWTTPSLKCIR |
| | GGGGSEPKSSDKTHTCPPCPAPELLRGPSVFLFPPKPKDTLYITRE |
| | PEVTCVVVDVSHEDPEVKFNWYVDGVEVHNAKTKPREEQYNST |
| | YRVVSVLTVLHQDWLNGKEYKCKVSNKARPAPIEKTISKAKGQP |
| | REPQVCTLPPSREEMTKNQVSLSCAVKGFYPSDIAVEWESNGQP |
| | ENNYKTTPPVLDSDGSFFLVSKLTVDKSRWQQGNVFSCSVMHE |
| | ALHNHYTQKSLSLSPG |

### EXAMPLES

The present disclosure, thus generally described, will be understood more readily by reference to the following Examples, which are provided by way of illustration and are not intended to be limiting of the present disclosure. The Examples are not intended to represent that the experiments below are all or the only experiments performed.

### Example 1

### Preparation of Materials, IL-15 Variants and Fusion Proteins

### 1.1 Preparation of Materials

Information on the commercially available materials used in the examples is provided in Table 1.

TABLE 1

| Materials | Vendor | Cat |
|---|---|---|
| Expi293F ™ Cells | Thermo Fisher | Cat. A14527 |
| ExpiFectamine293 transfection kit | Thermo Fisher | Cat. A14524 |
| Expi293F ™ expression medium | Thermo Fisher | Cat. A1435101 |
| Lipofectamine ™ 2000 Transfection Reagent | Thermo Fisher | Cat. 11668019 |
| FreeStyle ™ 293-F Cells | Thermo Fisher | Cat. R79007 |
| FreeStyle ™ 293 Expression Medium | Thermo Fisher | Cat. 12338002 |
| Opti-MEM | Thermo Fisher | Cat. 31985070 |
| Ni column | GE healthcare | Cat. 173712 |
| Protein A column | GE healthcare | Cat. 175438 |
| HPLC-SEC | TOSOH | Cat. 0008541 |
| NuPAGE4%-12% Bis-Tris Gel | Thermo Fisher | Cat. NP0322BOX |
| FITC mouse anti-human CD3 | BioLegend | 317306 |
| APC-Cy7 mouse anti-Human CD8 | BD Pharmingen | 557834 |
| PE mouse anti-Human CD56 | BioLegend | 318306 |
| APC mouse anti-human Ki-67 | BioLegend | 350514 |
| Alexa Fluor 647 mouse anti-Stat5 (pY694) | BD Biosciences | 562076 |
| FITC rat anti-mouse CD3 | Biolegend | 100204 |
| APC-Cy7 rat anti-mouse CD8a | BD Biosciences | 557654 |
| APC rat anti-mouse Ki-67 | Biolegend | 652406 |
| PE Rat anti-mouse CD335 | BD Pharmingen | 560757 |
| FITC mouse anti-human CD3 | BD Pharmingen | 556611 |
| PE-Cy7 mouse anti-Human CD8 | BD Pharmingen | Jan-27 |
| PerCP-cy5.5 mouse anti-KI-67 | BD Pharmingen | 561284 |
| Foxp3/Transcription Factor Staining Buffer Set | Ebioscience | 00-5523-00 |
| BD Phosflow ™ Fix Buffer I | BD Biosciences | BDB557870 |
| Phosflow Perm Buffer III | BD Biosciences | BDB558050 |
| Ficoll | Stem Cell | 7861 |
| ACK Lysing Buffer | GIBCO | A10492-01 |
| PE mouse anti-NHP CD45 | BD biosciences | 552833 |
| FITC mouse anti-human CD3 | BD biosciences | 556611 |
| APC-CY7 mouse anti-human CD8 | BD biosciences | 557834 |
| APC mouse anti-human CD16 | BD biosciences | 561248 |
| PE-CY7 mouse anti-human Ki-67 | BD biosciences | 561283 |
| Rat Anti-Mouse CD16/CD32 | BD biosciences | 553140 |
| Fixation/Permeabilization Concentrate | Invitrogen | 00-5123-43 |
| Fixation/Perm Diluent | Invitrogen | 00-5223-56 |
| Permeabilization Buffer 10X | Invitrogen | 00-833-56 |
| ACK Lysing Buffer | GIBCO | A10492-01 |
| Goat Anti-Human IgG | Southern Biotech | 2049-01 |
| Goat Anti-human IgG-Fc-biotin | Southern Biotech | SB-2049-08 |
| Streptavidin-HRP | Life Science | Life-SNN1004 |
| Mouse Anti-monkey IgG-HRP | Southern Biotech | SB-4700-05 |
| TMB | Solarbio | PR1200 |

### 1.2 Generation of IL-15 Variants, Fusion Proteins and BMKs

Construction of Expression Vectors for BMKs and IL15/IL15Rα-Fc Variants

Two heterodimeric IL15/IL15Rα-Fc fusion proteins, XENP24306 and XENP20818, disclosed in US20180118805A1 (the entire contents of which are incorporated herein by reference) were used as benchmarks in the following experiments. XENP24306 utilizes an IL-15 variant comprising D3N/E64Q/N65D mutation while XENP20818 utilizes a wild-type IL-15 protein. The DNA segments of XENP24306 (referred to as “W369-BK1”) and XNP29818 (referred to as “W369-BMK2”) were synthesized according to US20180118805A1 (Seq.382-387 and Seq.52-57) and sub-cloned into pcDNA3.4 expression vectors, then used in transfection for protein production.

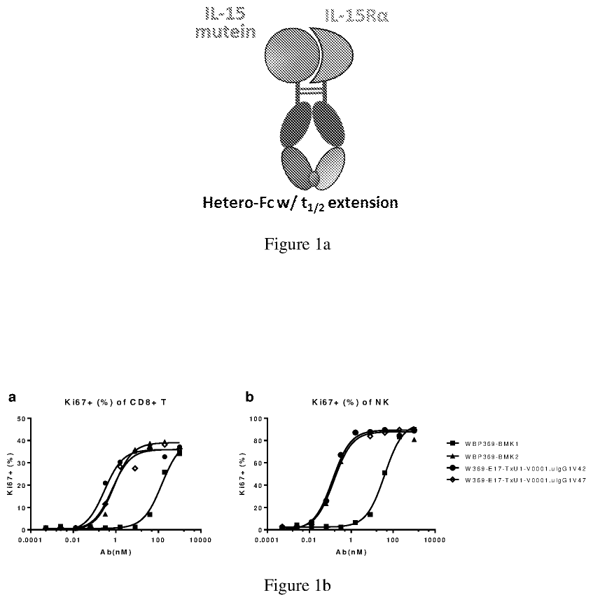

DNA fragments encoding the IL-15 variants listed in Table 2, IL15Rα, V47 Fc format, or V42 Fc format were also synthesized, sub-cloned into separate expression vectors and transfected for protein production. The structure of the generated heterodimeric fusion protein is as shown in FIG. 1a. For simplicity, the naming of these heterodimeric IL-15 fusion proteins may also be abbreviated into the format as “V00XX.uIgG1V42”, “V00XX.uIgG1V47” or “V00XX.uIgG1V805” throughout the specification.

TABLE 2
IL-15 heterodimeric fusion proteins

| | Mutations on |
|---|---|
| Variants ID | wild-type IL15 |
| W369-E17-TxU1-V0001.uIgG1V42/V47 | none |
| W369-E17-TxU1-V0003.uIgG1V42/V47 | S7N |
| W369-E17-TxU1-V0004.uIgG1V42/V47 | S7Q |
| W369-E17-TxU1-V0005.uIgG1V42/V47 | D8E |
| W369-E17-TxU1-V0006.uIgG1V42/V47 | D8S |
| W369-E17-TxU1-V0007.uIgG1V42/V47 | D8T |
| W369-E17-TxU1-V0008.uIgG1V42/V47 | D8Q |
| W369-E17-TxU1-V0009.uIgG1V42/V47 | K10Q |
| W369-E17-TxU1-V0010.uIgG1V42/V47 | D30E |
| W369-E17-TxU1-V0011.uIgG1V42/V47 | D30Q |
| W369-E17-TxU1-V0012.uIgG1V42/V47 | H32N |
| W369-E17-TxU1-V0014.uIgG1V42/V47 | D61Q |
| W369-E17-TxU1-V0015.uIgG1V42/V47 | N65Q |
| W369-E17-TxU1-V0016.uIgG1V42/V47 | I68A |
| W369-E17-TxU1-V0017.uIgG1V42/V47 | I68V |
| W369-E17-TxU1-V0018.uIgG1V42/V47 | L69A |
| W369-E17-TxU1-V0019.uIgG1V42/V47 | L69V |
| W369-E17-TxU1-V0020.uIgG1V42/V47 | Q108N |
| W369-E17-TxU1-V0021.uIgG1V42/V47 | N112Q |
| W369-E17-TxU1-V0058.uIgG1V42/V47 | S7N.K10Q.I68A |
| W369-E17-TxU1-V0059.uIgG1V42/V47 | K10Q.I68A.L69V |
| W369-E17-TxU1-V0060.uIgG1V42/V47 | S7N.K10Q.L69V |
| W369-E17-TxU1-V0061.uIgG1V42/V47 | S7N.I68A.L69V |
| W369-E17-TxU1-V0062.uIgG1V42/V47 | S7N.K10Q |
| W369-E17-TxU1-V0063.uIgG1V42/V47 | S7N.I68A |
| W369-E17-TxU1-V0064.uIgG1V42/V47 | S7N.L69V |
| W369-E17-TxU1-V0065.uIgG1V42/V47 | K10Q.I68A |
| W369-E17-TxU1-V0066.uIgG1V42/V47 | K10Q.L69V |
| W369-E17-TxU1-V0067.uIgG1V42/V47 | I68A.L69V |
| W369-E17-TxU1-V0068.uIgG1V42/V47 | I68F |
| W369-E17-TxU1-V0069.uIgG1V42/V47 | I68G |
| W369-E17-TxU1-V0070.uIgG1V42/V47 | I68L |
| W369-E17-TxU1-V0071.uIgG1V42/V47 | I68M |
| W369-E17-TxU1-V0088.uIgG1V42/V47 | I68K |
| W369-E17-TxU1-V0091.uIgG1V42/V47 | I68R |
| W369-E17-TxU1-V0092.uIgG1V42/V47 | I68Q |
| W327151-TXU1.P1-V0001.uIgG1v805 | Ala insertion between |
| | S7 and D8 |
| W327151-TXU1.P1-V0002.uIgG1v805 | Ala insertion between |
| | D8 and L9 |
| W327151-TXU1.P1-V0003.uIgG1v805 | Ala insertion between |
| | L9 and K10 |
| W327151-TXU1.P1-V0004.uIgG1v805 | Ala insertion between |
| | K10 and K11 |
| W327151-TXU1.P1-V0005.uIgG1v805 | Ala insertion between |
| | E64 and N65 |
| W327151-TXU1.P1-V0006.uIgG1v805 | Ala insertion between |
| | N65 and L66 |

Small Scale Protein Expression in HEK 293 Cells

Expi293 cells (Thermofisher, A14635) were transfected with plasmids constructed as described above, cultured for 5 days following the manufacturer suggested protocol. The supernatants were collected and analyzed by SDS-PAGE or filtered and used for target protein purification using Protein A column. The concentration of purified Fc-tagged proteins was determined by absorbance at 280 nm. The size and purity were tested by SDS-PAGE and SEC-HPLC, respectively, and then stored at −80° C.

Purity by SEC-HPLC

The purity of the heterodimeric proteins was tested by SEC-HPLC using Agilent 1260 Infinity HPLC. 50 μL of protein solution was injected on a TSKgel SuperSW3000 column using 50 mM sodium phosphate, 0.15 M NaCl, pH 7.0 buffer. The running time was 20 min. Peak retention times on the column were monitored at 280 nm. Data was analyzed using ChemStation software (V2.99.2.0).

### Example 2

### In Vitro Characterization

### 2.1 Ki-67 Proliferation Assays in Human/Mouse/Cyno PBMC

Heterodimeric IL15/IL15Rα-Fc fusion proteins containing different IL15 variants fused with V47, V42 or V805 Fc format were generated as described above and screened for variants with reduced potency. Briefly, human PBMCs were treated with wild type IL15 or IL-15 variants fused with V47/V42/V805 Fc format at the indicated concentrations. 4 days after treatment, the PBMCs were stained with anti-CD3, anti-CD8 and anti-CD56 to gate for CD8+ T cells (CD3+/CD8+) and NK cells (CD3−/CD56+), and then staining for intracellular Ki67 was performed using anti-Ki67 and Foxp3/Transcription Factor Staining Buffer Set. Lymphocytes were first gated on the basis of SSC and FSC. The lymphocytes were then gated based on CD3, CD8 and CD56 expression to identify CD8+T and NK cells. The percentage of Ki67 on CD8+T and NK cells was measured using FACS. The same procedure was performed for mouse spleen cells and Cyno PBMCs, respectively, and the percentage of Ki67 on CD8+T and NK cells was measured.

Wild type IL-15 induced strong proliferation of CD8+ T cells and NK cells. Upon treatment of human PBMC, the two Fc variants V42 and V47 containing a wild-type (wt) IL-15 moiety, i.e. V0001.uIgG1V42 and V0001.uIgG1V47, showed a comparable CD8+ T cell and NK cell proliferation, similar to W369-BMK2, as measured by Ki-67 proliferation (FIG. 1b), suggesting that additional mutations (“G236R/L328R” and “M252Y/S254T/T256E”) introduced to Fc v47 had little impact on the potency of IL-15 moiety.

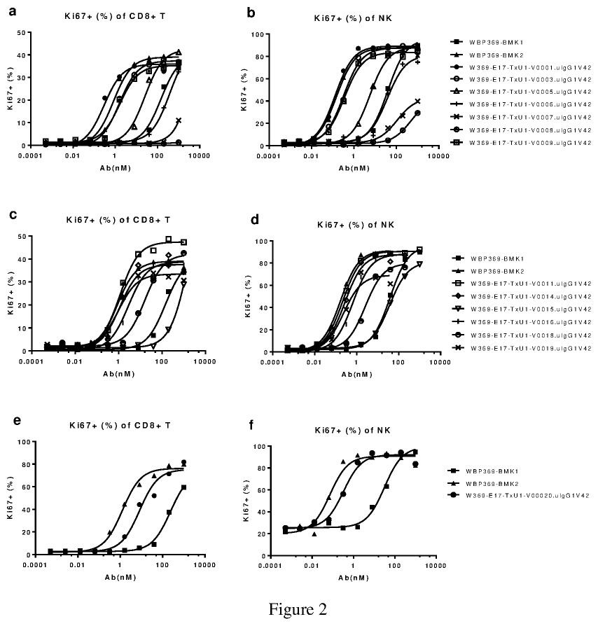

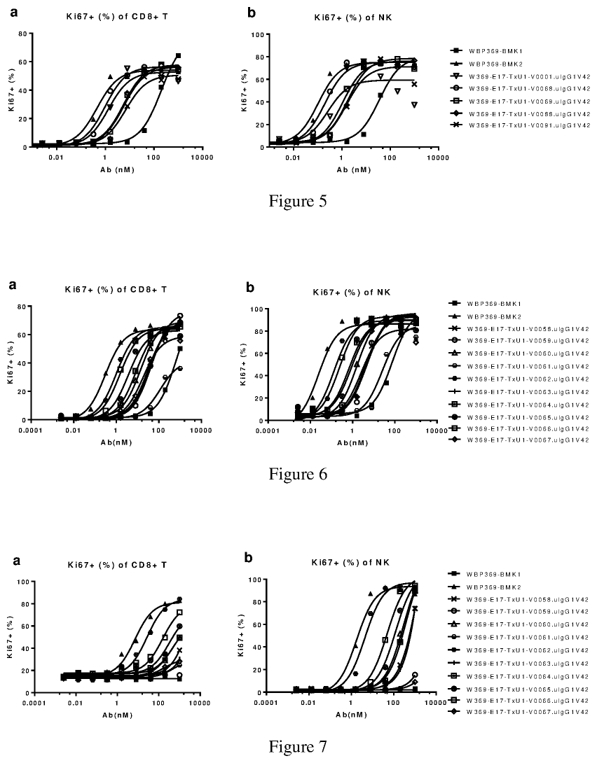

FIGS. 2 and 4-9 illustrate the percentage of Ki67 expression in CD8+ T cells and NK cells following treatment with different IL-15/Rα-Fc fusion proteins at indicated concentrations. Tables 3-4 show the EC50 values obtained by FACS. As shown in the result, the potency of V0003, V0005-V0009, V0011, V0014-V0016, V0018-V0020, V0058-V0069, V0088 and V0091 (both in V42 format or V47 format) were reduced to varying degrees compared to W369-BMK2 (IL-15 wt), in which V0007 and V0008 became almost inactive in stimulating CD8+T and NK cells.

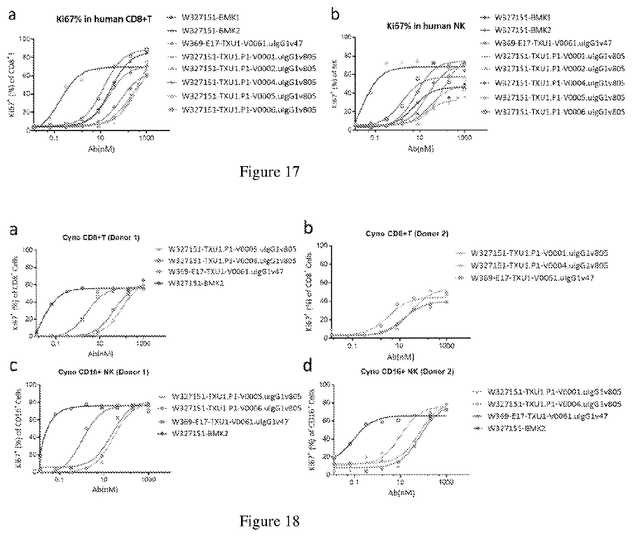

FIGS. 17-18 and Table 5 illustrate the corresponding result for fusion proteins in V805 Fc format.

TABLE 3A
Fold changes in EC50 for percentage of Ki67 expression on human and mouse
CD8+ T and NK cells following treatment with IL-15/Rα-Fc fusion proteins

| | | human | | | | mouse | | | |
|---|---|---|---|---|---|---|---|---|---|
| | | CD8+ T | | NK | | CD8+ T | | NK | |
| | Mutations | EC50 | Fold | EC50 | Fold | EC50 | Fold | EC50 | Fold |
| Ab | on IL15 | nM | reduced | nM | reduced | nM | reduced | nM | reduced |
| WBP369-BMK1 | D30N-E64Q- | 144.60 | 176 | 39.03 | 231 | inactive | inactive | inactive | inactive |
| | N65D | | | | | | | | |
| WBP369-BMK2 | none | 0.82 | \ | 0.17 | \ | 7.98 | \ | 2.16 | \ |
| W369-E17-TxU1-V0001.uIgG1V42 | none | 0.29 | \ | 0.13 | \ | 4.70 | \ | 1.86 | \ |
| W369-E17-TXU1-V0003.uIgG1V42 | S7N | 1.57 | 5 | 0.33 | 3 | 8.02 | 2 | 7.03 | 4 |
| W369-E17-TxU1-V0005.uIgG1V42 | DBE | 25.10 | 87 | 5.55 | 43 | inactive | inactive | very weak | very weak |
| W369-E17-TXU1-V0006.uIgG1V42 | D85 | 382.00 | 1317 | 36.68 | 282 | inactive | inactive | inactive | inactive |
| W369-E17-TxU1-V0007.uIgG1V42 | D8T | very weak | very weak | very weak | very weak | inactive | inactive | inactive | inactive |
| W369-E17-TxU1-V0008.uIgG1V42 | D8Q | inactive | inactive | very weak | very weak | inactive | inactive | inactive | inactive |
| W369-E17-TxU1-V0009.uIgG1V42 | K10Q | 1.64 | 6555 | 0.35 | 3 | 10.98 | 2 | 3.37 | 2 |
| W369-E17-TXU1-V0011.uIgG1V42 | D30Q | 1.16 | 4 | 0.21 | 2 | 22.68 | 5 | 31.96 | 17 |
| W369-E17-TXU1-V0014.uIgG1V42 | D61Q | 1.13 | 4 | 0.27 | 2 | 2.14 | 0.5 | 0.60 | 0.3 |
| W369-E17-TxU1-V0015.uIgG1V42 | N65Q | 1901.00 | 6555 | 37.45 | 288 | inactive | inactive | inactive | inactive |
| W369-E17-TxU1-V0016.uIgG1V42 | L68A | 2.80 | 10 | 0.65 | 5 | 62.24 | 13 | 74.16 | 40 |
| W369-E17-TxU1-V0018.uIgG1V42 | L69A | 16.21 | 56 | 2.54 | 20 | very weak | very weak | 695.50 | 374 |
| W369-E17-TxU1-V0019.uIgG1V42 | L69V | 0.98 | 3 | 0.23 | 2 | 23.52 | 5 | 30.68 | 16 |
| W369-E17-TxU1-V0020.uIgG1V42 | Q108N | 8.20 | 18 | 0.33 | 6 | very weak | very weak | 727.90 | 391 |

TABLE 3B
Fold changes in EC50 for percentage of Ki67 expression on human and mouse
5 CD8+ T and NK cells following treatment with IL-15/Rα-Fc fusion proteins

| | | human | | | | mouse | | | |
|---|---|---|---|---|---|---|---|---|---|
| | | CD8+ T | | NK | | CD8+ T | | NK | |
| | Mutations | EC50 | Fold | EC50 | Fold | EC50 | Fold | EC50 | Fold |
| Ab | on IL15 | nM | reduced | nM | reduced | nM | reduced | nM | reduced |
| WBP369-BMK1 | D30N-E64Q-N65D | 650.70 | 2169.00 | 74.34 | 2478.00 | inactive | inactive | inactive | inactive |
| WBP369-BMK2 | none | 0.30 | 1.00 | 0.03 | 1.00 | 7.11 | 1.00 | 1.77 | 1.00 |
| W369-E17-TXU1-V0058.uIgG1V42 | S7N.K10Q.I68A | 34.22 | 114.07 | 5.34 | 178.07 | Very weak | Very weak | Very weak | Very weak |
| W369-E17-TxU1-V0059.uIgG1V42 | K10Q.I68A.L69V | 45.39 | 151.30 | 4.94 | 164.73 | inactive | inactive | inactive | inactive |
| W369-E17-TxU1-V0060.uIgG1V42 | S7N.K10Q.L69V | 14.08 | 45.93 | 1.67 | 55.53 | Very weak | Very weak | 284.80 | 160.90 |
| W369-E17-TxU1-V0061.uIgG1V42 | S7N.I68A.L69V | 116.10 | 387.00 | 40.23 | 1341.00 | inactive | inactive | inactive | inactive |
| W369-E17-TxU1-V0062.uIgG1V42 | S7N.K10Q | 1.01 | 3.37 | 0.15 | 5.04 | 27.23 | 3.83 | 4.42 | 2.50 |
| W369-E17-TXU1-V0063.uIgG1V42 | S7N.I68A | 31.86 | 106.20 | 5.20 | 173.43 | inactive | inactive | Very weak | Very weak |
| W369-E17-TxU1-V0064.uIgG1V42 | S7N.L69V | 8.44 | 28.15 | 1.01 | 33.67 | Very weak | Very weak | 396.10 | 223.79 |
| W369-E17-TXU1-V0065.uIgG1V42 | K10Q.I68A | 3.70 | 12.32 | 0.85 | 28.28 | 321.20 | 45.18 | 122.00 | 68.93 |
| W369-E17-TxU1-V0066.uIgG1V42 | K10Q.L69V | 1.50 | 5.00 | 0.26 | 8.59 | 152.60 | 21.46 | 48.90 | 27.63 |
| W369-E17-TXU1-V0067.uIgG1V42 | I68A.L69V | 18.03 | 60.10 | 4.01 | 133.70 | inactive | inactive | inactive | inactive |

TABLE 4
Fold changes in EC50 for percentage of Ki67 expression on human CD8+
T and NK cells following treatment with IL-15/Rα-Fc fusion proteins

| | | human | | | |
|---|---|---|---|---|---|
| | | CD8+ T | | NK | |
| Ab | Mutations on IL15 | EC50 nM | Fold reduced | EC50 nM | Fold reduced |
| WBP369-BMK1 | D30N-E64Q-N65D | 202.90 | 441.09 | 35.49 | 354.90 |
| WBP369-BMK2 | none | 0.46 | 1.00 | 0.10 | 1.00 |
| W369-E17-TxU1-V0001.uIgG1V42 | WT | 1.34 | 2.91 | 0.26 | 2.60 |
| W369-E17-TxU1-V0068.uIgG1V42 | I68F | 0.98 | 2.13 | 0.20 | 2.00 |
| W369-E17-TxU1-V0069.uIgG1V42 | I68G | 5.53 | 12.02 | 1.06 | 10.60 |
| W369-E17-TxU1-V0088.uIgG1V42 | I68K | 5.72 | 12.43 | 1.75 | 17.50 |
| W369-E17-TxU1-V0091.uIgG1V42 | I68R | 6.75 | 14.67 | 1.54 | 15.40 |

TABLE 5A
Fold changes in EC50 for percentage of Ki67 expression on human CD8+
T and NK cells following treatment with IL-15/Rα-Fc fusion proteins

| | | human | | | |
|---|---|---|---|---|---|
| | | CD8+ T | | NK | |
| Ab | Mutations IL15 | EC50 nM | Fold reduced | EC50 nM | Fold reduced |
| WBP369-BMK1 | D30N-E64Q-N65D | 28.47 | 154.7 | 5.115 | 243.6 |
| WBP369-BMK2 | None | 0.184 | 1.00 | 0.021 | 1.00 |
| W369-E17-TxU1-V0061.uIgG1V42 | S7N.I68A.L69V | 76.22 | 414 | 20.98 | 999 |
| W327151-TXU1.P1-V0001.uIgG1v805 | Insertion A8 (S7-D8) | 337.6 | 1835 | 29.19 | 1390 |
| W327151-TXU1.P1-V0002.uIgG1v805 | Insertion A9 (D8-L9) | 174.3 | 947 | 19.13 | 911 |
| W327151-TXU1.P1-V0004.uIgG1v805 | Insertion A11 (K10-K11) | 22.51 | 122.3 | 6.716 | 320 |
| W327151-TXU1.P1-V0005.uIgG1v805 | Insertion A65 (E64-N65) | 352.0 | 1913 | 105.7 | 5033 |
| W327151-TXU1.P1-V0006.uIgG1v805 | Insertion 66 (N65-L66) | 13.52 | 73.5 | 1.577 | 75.1 |

TABLE 5B
Fold changes in EC50 for percentage of Ki67 expression on cyno CD8+ T
and NK cells following treatment with IL-15/Rα-Fc fusion proteins

| | Cyno Donor 1 | | | | Cyno Donor 2 | | | |
|---|---|---|---|---|---|---|---|---|
| | CD8+ T | | NK | | CD8+ T | | NK | |
| | EC50 | Fold | EC50 | Fold | EC50 | Fold | EC50 | Fold |
| Ab | nM | reduced | nM | reduced | nM | reduced | nM | reduced |
| WBP369-BMK2 | | | 0.09 | 1 | 0.017 | 1 | 0.008 | 1 |
| W369-E17-TxU1-V0061.uIgG1V42 | 14.76 | 164 | 71.26 | 792 | 46.77 | 2751 | 22.77 | 2846 |
| W327151-TXU1.P1-V0001.uIgG1v805 | 29.21 | 325 | 65.57 | 729 | | | | |
| W327151-TXU1.P1-V0002.uIgG1v805 | | | | | | | | |
| W327151-TXU1.P1-V0004.uIgG1v805 | 3.374 | 37 | 10.35 | 115 | | | | |
| W327151-TXU1.P1-V0005.uIgG1v805 | | | | | 100.2 | 5894 | 26.33 | 3291 |
| W327151-TXU1.P1-V0006.uIgG1v805 | | | | | 2.593 | 153 | 1.04 | 130 |

2.2 pSTAT5 Proliferation Assay

Transpresentation of IL-15 and IL-15Rα drives phosphorylation of STAT5 and subsequent proliferation of NK and T cells. Accordingly, CD8+T and NK cells were analyzed for STAT5 phosphorylation following 30 minutes incubation with the indicated IL-15/Rα-Fc variants. At the end of the treatment, PBMC were immediately fixed by BD Phosflow. Fix Buffer I and then incubated with pre-chilled BD Phosflow. Perm Buffer III for permeabilization. After incubation, cells were stained with anti-CD3, anti-CD56 and anti-pSTAT5 for 30-40 minutes at room temperature. Lymphocytes were first gated on the basis of SSC and FSC, and then gated based on CD3 and CD56 expression to identify NK cells. Finally, the phosphorylation level of STAT5 in the various cell populations was determined.

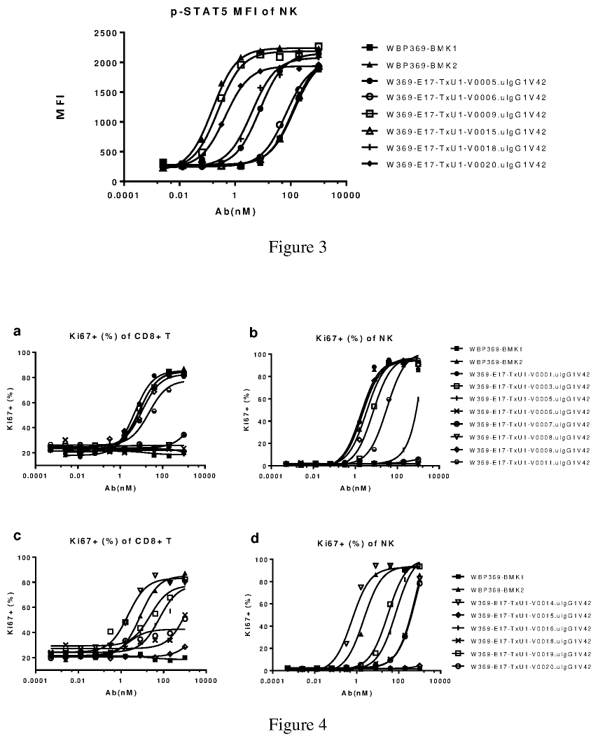

The STAT5 phosphorylation of NK cells following treatment with the V0005, V0006, V0009, V0015, V0018 and V0020 are reduced to varying degrees (FIG. 3), fairly consistent with NK proliferation measured by Ki-67 assay.

### 2.3 Structure Modeling

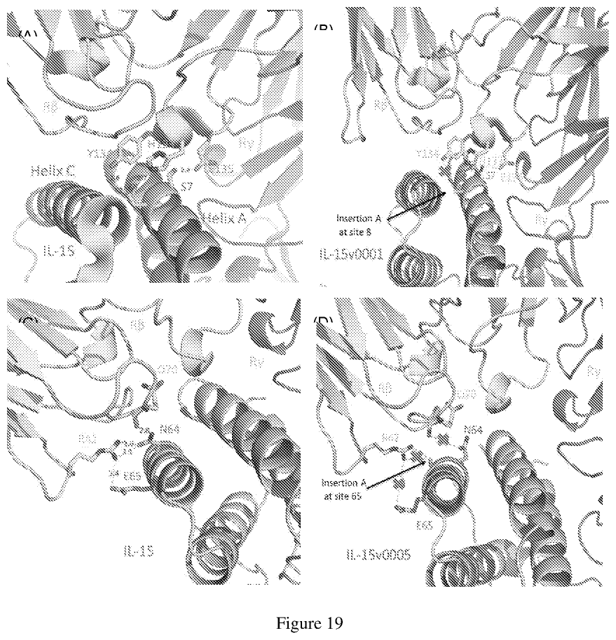

The homology models of IL-15 variants were constructed in Discovery Studio using the solved IL-15 crystal structure (PDB: 4GS7) as the template. 10 models were generated, and the best pose was picked based on the DOPE scores in Discovery Studio. The interaction model to its receptors of α, β and γ was obtained by superimposing the IL-15 variant model on the available complex crystal structure (PDB: 4GS7) in PyMol. All structures were visualized and analyzed in PyMol. The result is shown in FIG. 19.

The IL-15 quaternary complex contains its private IL-15 Rα and the IL-2-shared signaling receptors IL-2Rβ and Rγ. The IL-15 structure contains four helices (A, B, C and D) connected by different loops (Ring, Aaron M., et al. “Mechanistic and structural insight into the functional dichotomy between IL-2 and IL-15.” Nature immunology 13.12 (2012): 1187-1195). Helix A is composed of amino acids at positions from 1 to 17 of SEQ ID NO: 1 (‘NWVNVISDLKKIEDLIQ’); Helix B is composed of amino acids at positions from 32 to 53 of SEQ ID NO: 1 (‘HPSCKVTAMKCFLLELQVISLE’); Helix C is composed of amino acids at positions from 57 to 77 of SEQ ID NO: 1 (‘ASIHDTVENLIILANNSLSSN’); and Helix D is composed of amino acids at positions from 95 to 113 of SEQ ID NO: 1 (‘IKEFLQSFVHIVQMFINT’).

It has been found that the N-terminal helix (Helix A) interacts with both IL-2Rβ and Rγ, the third helix (Helix C) interacts with IL-2Rβ, and the C-terminal helix (Helix D) interacts with Rγ (Ring, Aaron M., et al. “Mechanistic and structural insight into the functional dichotomy between IL-2 and IL-15.” Nature immunology 13.12 (2012): 1187-1195). Specifically, N65 forms three hydrogen bonds to R42, Q70, and Y134 of IL-2Rβ, D8 forms two hydrogen bonds to H133 and Y134 of IL-2Rβ; K10 forms a salt-bridge to E136 of IL-2Rβ; D61 forms a salt-bridge to K71 of IL-2Rβ; S7 forms a hydrogen bond to E136 of IL-2Rβ; K11 presents the aliphatic portion of its side-chain for van der Waals interactions with H133 of IL-2Rβ ; I68 and L69 form van der Waals interactions with T73 and V75 of IL-2Rβ; I68 and L69 form van der Waals interactions with T73 and V75 of IL-2Rβ.

The insertion of an additional Alanine residue at key positions on IL-15 that are involved in the receptor binding, may disrupt or weaken the original key interactions (e.g. hydrogen bond, salt bridge, etc.) and reduce the binding affinity of IL-15 as well as its corresponding biological potency (e.g. the capability of stimulating T/NK cell proliferation). FIG. 19 showed two examples. The model of IL-15 variant (v0001) suggested that the insertion of Alanine at site 8 could potentially disrupt the original two hydrogen bonds formed between D8 (IL-15 Helix A) and H133/Y134 (receptor β), and weaken the original hydrogen bond formed between S7 (IL-15 helix A) and E135 (receptor β). Similarly, the model of IL-15 variant (v0005) suggested that the insertion of Alanine at site 65 could potentially disrupt the hydrogen bonds between N64 (IL-15 Helix C) and R42/Q70 (receptor β) and break the salt bridge between E65 (IL-15 Helix C) and R42 (receptor β).

The insertion of other amino acids into Helix A/C, such as non-polar amino acids (Gly, Val, Leu, Be, Met, Trp, Phe, Pro), polar amino acids (Ser, Thr, Cys, Tyr, Asn, Gln), and charged amino acids (Asp, Glu, Lys, Arg, His), and even unnatural amino acids could also affect the IL-15 potency. It could be explained by two potential mechanisms: (A) Destabilizing the Helix A/C and thus affecting the binding; (B) Pushing the position of the rest residues in the Helix A/C, thus mismatching key interactions with original amino acids.

### Example 3

### In Vivo Characterization

Cynomolgus monkey was selected for investigation since cynomolgus monkey was proven to be an appropriate species for the PK/PD and toxicity study. The study was designed following CFDA's Good Laboratory Practices Regulations for Non-clinical Drug Evaluation.

Monkey was individually housed in stainless steel, slat floor cages. Animal room was maintained on a 12/12 hours light/dark cycle (light/dark cycle was interrupted for blood sampling at 8 h post-dose on Day 0), within a temperature range of 16 to 26° C. (60.8 to 78.8° F.), and a relative humidity range of 40 to 70%. Feed supplied by Beijing KeAoXieLi Feed Company Ltd was provided daily in amounts appropriate for the size and age of the animals. The monkey was fed twice daily at approximately 6 hours apart. The first feeding on dosing day was provided at approximately 0.5 hours post-dose. Any food remaining from the previous day was discarded prior to feed. Water was provided by a water-feed autodrinker. No contaminants were known to be in the diet or water in quantities that was expected to interfere with the outcome of this study.

Four male and four female monkeys, 2 to 3 years old, with a body weight ranging from 2.90 kg to 4.01 kg at initiation of dosing, were enrolled. At the start of the pre-dose period, each monkey was identified by collar tag and as indicated on its cage card. After assignment to dosage groups, each monkey was assigned a unique test number, which was indicated on its cage card together with its collar tag.

The test articles, identified as W369-BMK1, W369-BMK2, W369-E17-TxU1-V0006.uIgG1V47 and W369-E17-TxU1-V0061.uIgG1V47, were generated and characterized as described above.

### 3.1 In Vivo Study Design

The quarantine period was 29 days (prior to pre-dose period). The pre-dose period was 27 days prior to the initiation of dosing. Intravenous administration is the intended clinical route of administration.

Cynomolgus monkey was selected on the basis of pre-dose evaluations and randomly assigned to groups (n=2, one male & one female) based on body weight. Monkey was injected intravenously with W369-BMK1 (0.6 mpk), W369-BMK2 (0.3 mpk), W369-E17-TxU1-V0006.uIgG1V47 (0.6 mpk) and W369-E17-TxU1-V0061.uIgG1V47 (0.6 mpk) on Day 0, the injection site was marked using permanent ink and the tissue of the injection area was monitored closely during the study.

The following observations and measurements were made on all monkeys as indicated.

### Mortality and Clinical Observations

Mortality checks were performed once daily after release from quarantine. Clinical observations were performed twice daily during the pre-dose and non-dosing days.

On the dosing day, clinical observations were recorded prior to dosing and at 0.5, 1 and 4 hours after the dosing was completed. Clinical observations were included, but not limited to, appearance, behavioral activity, gland secretion, respiration, stool properties and local reactions of injection site.

### Body Weight

Determined once during the pre-dose period and twice weekly during first 2 weeks of the study.

### Food Consumption

Determined twice daily prior to dose. Determined daily during the first 2 weeks of study, except during fasting periods (estimated as 0, 25, 50, 75 or 100% of the total daily given amount).

### Body Temperature

Body temperature was measured at the rectum at pre-dose (Day 0) and 4 h, Day1 (24 h), Day2 (48 h), Day3 (72 h), Day4 (96 h), Day5 (120 h), Day6 (144 h), Day7 (I68 h), Day8 (192 h), Day10 (240 h), Day12 (288 h) and Day14 (336 h) after dosing.

### Hematology and Clinical Biochemistry

Blood was collected at pre-dose (Day −4) and 4 h, Day2 (48 h), Day4 (96 h), Day6 (144 h), Day8 (192 h), Day12 (264 h), Day14 (336 h) after dosing from all surviving monkeys for hematology and clinical chemistry evaluation. 0.5 mL blood was collected into tubes containing EDTA-K2 for hematology and 1.0 mL blood was collected into tubes without additive for serum chemistry determinations.

Hematology: pre-dose (Day −4), 4 h, Day2 (48 h), Day4 (96 h), Day6 (144 h), Day8 (192 h), Day12 (264 h), Day14 (336 h) after dosing Clinical Chemistry: pre-dose (Day −4), 4 h, Day2 (48 h), Day4 (96 h), Day6 (144 h), Day8 (192 h), Day12 (264 h), Day14 (336 h) after dosing

### 3.2 Pharmacokinetics (PK)

### 3.2.1 Blood Sampling

The systemic exposure to the test articles was assessed in all monkeys. Blood samples were collected at pre-dose (Day −4) and 4 h, Day1 (24 h), Day3 (72 h), Day5 (120 h), Day7 (I68 h), Day10 (240 h), Day12 (288 h), Day14 (336 h), Day19 (456 h), Day26 (624 h) after dosing. At each time point, approximately 0.5 mL blood was collected from the fundus venous plexus to harvest serum. Extreme care was taken during blood collection. Blood samples were collected into tubes with additive EDTA and placed on ice until they were processed. All samples were processed within 2 hours of collection.

For immunology analysis, approximately 0.5 mL blood was collected to harvest serum from all surviving monkeys at pre-dose (Day −4) and Day 7 (I68 h), Day 14 (336 h), Day 26 (624 h) after dosing. Serum was harvested by centrifugation at 3,500 rpm and 4 C for 15 minutes and stored frozen at approximately −70 C or lower until transferred.

### 3.2.2 Measurement of Drug Serum Concentration by ELISA

Briefly, ELISA plate was coated with 1 μg/mL goat anti-human IgG Fc (Southern Biotech, L1518-TB09). The Fc-Fc method was described as follows: After washing and blocking, serial diluted serum samples were added and then biotin labeled goat anti-human IgG Fc (Southern Biotech, SB-2049-08) was used as detection antibody. HRP-Streptavidin (Life-SNN1004) and TMB substrate (Solarbio, PR1200) were used for color development. The absorbance of the wells was measured at (450-540) nm with a multiwall plate reader (SpectraMax® M5e). Standard curve was generated according to the standard samples, and serum samples were analyzed with SoftMax. The result was shown in FIG. 10.

### 3.2.3 Measurement of Anti-Drug Antibody (ADA) by ELISA

ELISA plate was coated with 1 μg/mL W369-BMK1, W369-BMK2, W369-E17-TxU1-V0006.uIgG1V47 and W369-E17-TxU1-V0061.uIgG1V47. After washing and blocking, serial diluted serum samples were added and then mouse anti-monkey IgG-HRP (SB-4700-05) was used as detection antibody. Then TMB substrate (Solarbio, PR1200) were used for color development. The absorbance was measured as above. Serum of pre-dosing was used as baseline. The result was shown in Table 7.

### 3.2.4 Data Analyses

The data was evaluated for drug-related effects by making comparisons of individual value, and between intervals where appropriate. The significance of any findings was determined by the responsible investigator based on prior experience of the scientists involved.

The serum concentration was subjected to a non-compartmental pharmacokinetic analysis by using the Phoenix WinNonlin software (version 8.1, Pharsight, Mountain View, Calif.). The linear/log trapezoidal rule was applied in obtaining the PK parameters, data was expressed with Mean±SD.

The pharmacokinetics of test articles was evaluated in the cynomolgus monkeys. As shown in FIG. 10 & Table 6, potency reduction in BMK1, V0006 and V0061 obviously led to extended serum half-life compared to wild type IL-15 (BMK2). Specifically, the lower the in vitro potency (V0061<V0006<BMK1<BMK2), the longer the serum half-life (V0061>V0006>BMK1>BMK2). Consistently, lower potency also increased the Cₘₐₓ and drug exposure (AUC), without causing toxicity as measured in parallel studies or ADA production (Table 7). Prolonged PK profile will likely contribute to extended drug exposure and improved pharmacodynamics.

TABLE 6
PK parameters determined by noncompartmental analysis (WinNonlin)

| | W369- | W369- | W369-E17-TxU1- | W369-E17-TxU1- |
|---|---|---|---|---|
| Compound | BMK1 | BMK2 | V0006.uIgG1V47 | V00061.uIgG1V47 |
| t_{1/2} (h) | 162^{a} | 11.2^{b} | 170^{a} | 195^{a} |
| Cₘₐₓ (μg/mL) | 12.7 | 3.44 | 13.0 | 14.3 |
| AUC₀₋ₜ (h*μg/mL) | 812 | 72.5 | 1031 | 1060 |
| Cl_obs (mL/day/kg) | 17.6 | 90.0 | 13.4 | 11.8 |
| MRTINF_obs (h) | 93.3 | 19.2 | 146 | 219 |
| Vss_obs (mL/kg) | 67.9 | 71.3 | 81.2 | 107 |

^{a}the t ½ were calculated with the time point of 7 d-19 d.
^{b}the t ½ were calculated with the time point of 4 h-24 h.

TABLE 7
ADA production determined by ELISA

| | W369- | | W369- | | W369-E17-TxU1- | | W369-E17-TxU1- | |
|---|---|---|---|---|---|---|---|---|
| Compounds | BMK1 | | BMK2 | | V0006.uIgG1V47 | | V00061.uIgG1V47 | |
| Animal No. | 1001 | 1501 | 2001 | 2501 | 3001 | 3501 | 4001 | 4501 |
| Day 7 | − | ++ | + | + | − | + | − | − |
| Day 14 | − | + | + | − | − | + | − | − |
| Day 26 | − | − | − | − | − | + | − | +++ |

ADA Criteria: OD ratio (S/N) <2, −; 2~5, +; 5~10, ++; ≥10, +++.

### 3.3 Pharmacodynamics (PD)

### 3.3.1 PBMC Collection

The lymphocytes proliferation to the test article was assessed in all monkeys. Blood samples were collected at pre-dose (Day −4) and 4 h, Day1 (24 h), Day2 (48 h), Day3 (72 h), Day4 (96 h), Day5 (120 h), Day6 (144 h), Day7 (I68 h), Day8 (192 h), Day10 (240 h), Day12 (288 h), Day14 (336 h), Day19 (456 h), Day26 (624 h) after dosing. At each time point, approximately 1.0 mL blood was collected from the fundus venous plexus to harvest peripheral blood mononuclear cells (PBMC).

Blood samples was treated with 10 mL ACK lysis for 5 min at room temperature and centrifuged at 450 g for 5 min to collect the bottom cells, as PBMCs. After one wash with 10 ml PBS, the cells were resuspended in 0.4 ml PBS, stained for cell surface markers of CD45, CD3, CD8, CD16, or additionally with intracellular marker Ki-67, and subjected to flow cytometry analysis.

### 3.3.2 Flow Cytometry

200 μl PBMCs prepared as above were analyzed by cell surface staining after 2 μL purified/rat anti-mouse CD16/CD32 (2.4G2) to block non-specific binding. Each fluorochrome-conjugated monoclonal antibody in 2 μl was added, mixed evenly and let them stand for staining in dark for 30 minutes. PBMCs were washed by PBMC once, and resuspended in 100 μl PBS. Then 100 μl was performed on a FACS Calibur flow cytometer (Cantoll, BD), data were analyzed using Flowjo Software.

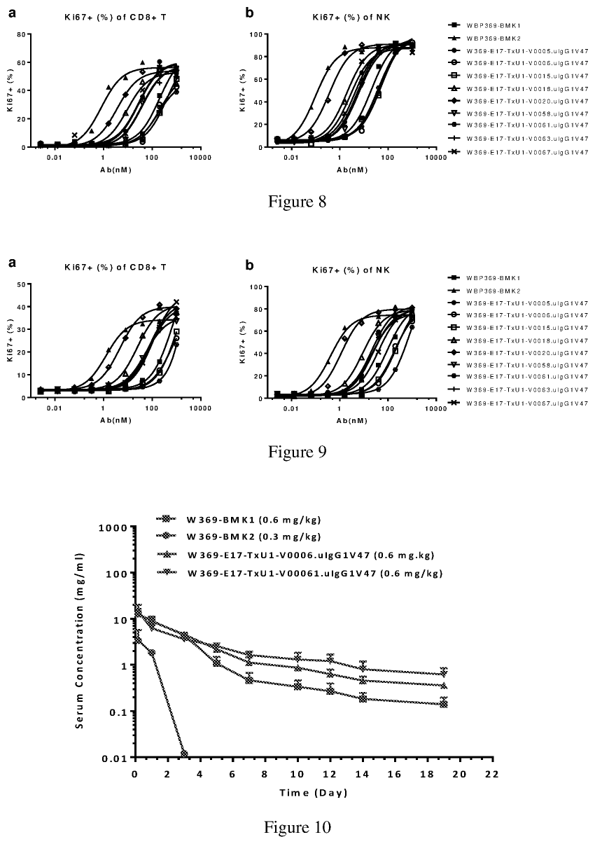

For Ki-67 staining, another 100 μl stained PBMCs were penetrated by Foxp3 transcription staining buffer for 30 min. PE-CY7 mouse anti-human Ki-67 in 2 μl was added to staining for 30 min, then performed on a FACS Calibur flow cytometer. The potency of the indicated IL-15 fusion proteins on proliferating CD8+T and NK cells of human (FIG. 8) and cynomolgus monkeys (FIG. 9) were measured by Ki-67 assay following incubation of PBMC with these variants.

Of notion, the degree of potency reduction for these variants were consistent in human and cyno PBMC, suggesting cyno monkeys would serve as a valid surrogate model to study the PK/PD effects of these IL-15 variants in humans.

### 3.3.3 Cytokine Release Detection

Blood samples were collected at pre-dose (Day −4) and 1 h, 4 h, 8 h, Day1 (24 h), Day3 (72 h), Day7 (I68 h), Day10 (240 h), Day14 (336 h) after dosing. At each time point, approximately 0.2 mL blood was collected from the fundus venous plexus to harvest serum. Serum cytokines were measured using NHP CBA Th1/Th2 cytokine kits (BD Biosciences).

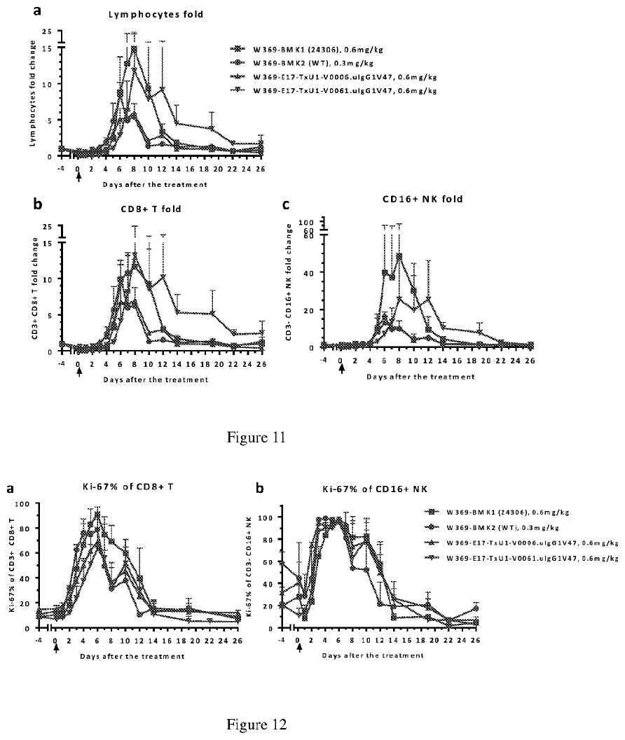

IL-15 is known to exhibit broad activity on various immune cells and induces lympho-expansion in vitro and in vivo. As shown in FIG. 11a, treatment of cyno monkeys with wild type IL-15 (BMK2) led to an earlier (peaked at day 6) but much lower lymphocyte proliferation (maximally ˜8.4 fold increase comparing with pre-dose). In contrast, all other three IL-15 variants with reduced potency showed a delayed (peaked around day 8 or later) but usually higher level of lympho-expansion (average fold of BMK1, V0006 and V0061 were 14.7, 5.8 and 12, respectively). The same trend was observed for NK and CD8+ T cells (FIG. 11 b, c): for example, in terms of NK cell proliferation, the peak for BMK2 (wt IL-15) was 10-fold at day 6, but the averaged fold increase of BMK1, V0006 and V0061 were 48, 10 and 25 at day 8, respectively. Specifically, the IL-15 variant with most potency reduction, v0061, showed a most pronounced and extended PD effect on NK and CD8+ T cells, leading to several fold higher levels of NK/T counts than pre-dose level beyond day 14, when in most other treatment groups, the NK/T cell counts have dropped to pre-dose level.

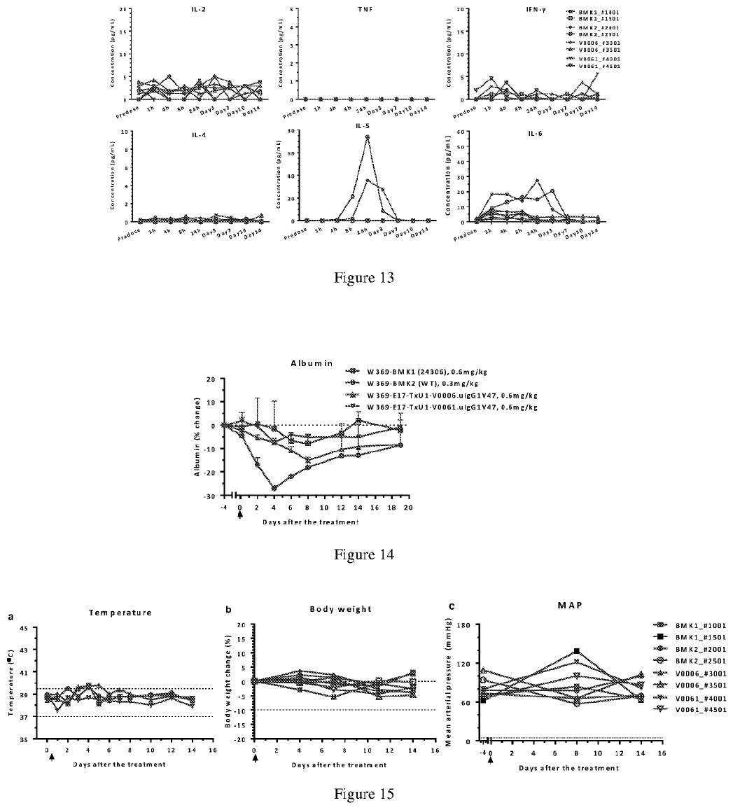

Consistent with the above observations, as shown in FIG. 12, the Ki-67 proliferation marker of NK/CD8+T peaked earlier (day 4) in BMK2 (wt IL-15) treated animals than potency reduced variants (day6, for BMK1, v0006, v0061).

### Example 4

### Exploratory Toxicity Studies

To investigate the impact of potency reduced IL-15 variants on the previously observed toxicity for this proinflammatory cytokine, a series of exploratory toxicity studies were performed.

### 4.1 Inflammatory Cytokine Release

As shown in FIG. 13, no obvious change in IL-2, TNF, IFN-γ (Th1) and IL-4 (Th2) was observed for all groups after the administration. In BMK2 group, transient increase of IL-5 at 8-24 h (Neutrophile) and IL-6 at 1-72 h (inflammation) was observed after administration, consistent with the previous findings in toxicity. All potency reduced IL-15 variants showed no distinct inflammatory cytokine production

### 4.2 Clinical Observation

Monkeys receiving BMK2 (wt IL-15) showed a significant reduction in albumin during the first 4 days (FIG. 14), indicating potential liver injury. In contrast, none of the three potency reduced variants caused such pronounced change. Moreover, there were no significant differences in mean body weights or any other dose-related clinical or behavioral observations among the groups (FIG. 15).

### 4.3 Hematology and Blood Biochemistry

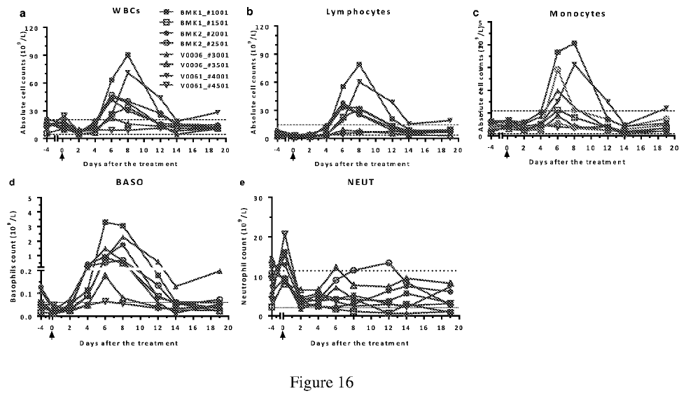

As shown in FIG. 16, except the expected observation of IL-15 induced transient lympho-expansion (counts in WBCs, lymphocytes, monocytes and basophile), for all treatment groups no other abnormalities were observed. The blood biochemistry readouts were also within the normal range (Table 8), suggesting no obvious toxicity issues for all treatment groups.

TABLE 8
Representative hematology characteristics of cynomolgus monkeys following one dose of IL-15 administration (pre-dose and day 19)

| | BMK1, | | BMK2, | | V0006, | | V00061, | |
|---|---|---|---|---|---|---|---|---|
| | 0.6 mg/kg | | 0.3 mg/kg | | 0.6 mg/kg | | 0.6 mg/kg | |
| Parameter | Pre-dose | Day 19 | Pre-dose | Day 19 | Pre-dose | Day 19 | Pre-dose | Day 19 |
| RBC count (10⁶/μL) | 5.4 ± 0.04 | 4.6 ± 0.20 | 5.8 ± 0.06 | 5.3 ± 0.40 | 5.8 ± 0.45 | 4.9 ± 0.30 | 6.3 ± 0.56 | 5.3 ± 0.20 |
| Hemoglobin (g/L) | 12.5 ± 0.6 | 10.6 ± 0.4 | 12.6 ± 0.6 | 11.5 ± 0.9 | 12.1 ± 0.1 | 10.0 ± 0.3 | 13.6 ± 1.6 | 11.4 ± 0.9 |
| Platelets (10³/μL) | 305 ± 46 | 411 ± 34..0 | 435 ± 6 | 523 ± 42 | 349 ± 55 | 367 ± 56 | 379 ± 29 | 339 ± 53 |
| ALT (U/L) | 30.9 ± 0.5 | 33.8 ± 5.0 | 32.4 ± 5.2 | 46.3 ± 23.6 | 45.3 ± 11.3 | 62.9 ± 22.1 | 46.7 ± 10.7 | 78.6 ± 35.3 |
| AST (U/L) | 38.0 ± 8.3 | 34.1 ± 4.9 | 53.6 ± 4.4 | 42.6 ± 0.6 | 40.3 ± 0.8 | 47.4 ± 4.7 | 54.6 ± 2.2 | 46.2 ± 6.9 |
| ALP (U/L) | 283 ± 187 | 233 ± 135 | 424 ± 39 | 374 ± 55 | 299 ± 44 | 375 ± 48 | 246 ± 124 | 217 ± 113 |
| TIBL (μmol/L) | 1.8 ± 0.10 | 2.7 ± 0.50 | 1.9 ± 0.25 | 2.4 ± 0.40 | 1.7 ± 0.30 | 2.5 ± 0.40 | 1.6 ± 0.55 | 2.3 ± 0.50 |
| CRE (μmol/L) | 65.3 ± 0.4 | 66.6 ± 4.0 | 63.4 ± 0.4 | 60.7 ± 2.1 | 65.2 ± 2.8 | 57.6 ± 0.8 | 80.6 ± 12.1 | 75.6 ± 2.7 |
| Urea (μmol/L) | 6.2 ± 0.47 | 6.7 ± 0.10 | 6.8 ± 0.17 | 5.9 ± 0.10 | 5.6 ± 0.02 | 5.1 ± 0.30 | 7.6 ± 0.69 | 8.2 ± 0.20 |

Those skilled in the art will further appreciate that the present invention may be embodied in other specific forms without departing from the spirit or central attributes thereof. In that the foregoing description of the present disclosure provides only exemplary embodiments thereof, it is to be understood that other variations are contemplated as being within the scope of the present invention. Accordingly, the present invention is not limited to the particular embodiments that have been described in detail herein. Rather, reference should be made to the appended claims as indicative of the scope and content of the invention.

1-38. (canceled)

39. An IL-15 variant, wherein the IL-15 variant has a lower potency in stimulating immune cell proliferation compared to wild-type IL-15, and has an amino acid sequence comprising one or more of the following mutations compared to the amino acid sequence as set forth in SEQ ID NO: 1:
(a) D8S, D8E, D8T, D8Q, D30E, D30Q, D61Q, N65Q, Q108N, a substitution at position 7, a substitution at position 10, a substitution at position 32, a substitution at position 68, a substitution at position 69, a substitution at position 112;
(b) an insertion in the amino acid sequence of helix A, wherein the amino acid sequence of helix A consists of amino acids at positions 1 to 17 of SEQ ID No: 1; and
(c) an insertion in the amino acid sequence of helix C, wherein the amino acid sequence of helix C consists of amino acids at positions 57 to 77 of SEQ ID No: 1.

42. The IL-15 variant of claim 39, wherein the IL-15 variant comprises one or more substitutions selected from D8S, S7N, S7Q, S7A, S7D, S7E, S7F, S7G, S7H, S7I, S7K, S7L, S7M, S7P, S7R, S7T, S7V, S7W, S7Y, D8E, D8T, D8Q, K10Q, D30E, D30Q, H32N, D61Q, N65Q, I68A, I68V, I68F, I68G, I68K, I68R, I68L, I68M, I68Q, I68D, I68E, I68H, I68N, I68P, I68S, I68T, I68W, I68Y, L69A, L69V, L69D, L69E, L69F, L69G, L69H, L69I, L69K, L69M, L69N, L69P, L69Q, L69R, L69S, L69T, L69W, L69Y, Q108N, and N112Q.

41. The IL-15 variant of claim 39, wherein the IL-15 variant comprises one or more substitution(s) selected from S7N, D8E, D8S, D8T, D8Q, K10Q, D30Q, D61Q, N65Q, I68A, I68F, I68G, I68K, I68R, L69A, L69V, and Q108N.

42. The IL-15 variant of claim 39, wherein the IL-15 variant comprises:
(a) a double substitution of S7N/K10Q, S7N/I68A, S7N/L69V, K10Q/I68A, K10Q/L69V, or I68A/L69V;
(b) a triple substitution of S7N/K10Q/I68A, K10Q/I68A/L69V, S7N/K10Q/L69V, or S7N/I68A/L69V; and/or
(c) the amino acid sequence of amino acids 1-114 of SEQ ID No: 19, 9, 7, 11, 13, 15, 17, 21, or 23.

43. The IL-15 variant of claim 39, wherein said insertion is a one amino acid insertion that disrupts the hydrogen bonding, salt bridge, and/or van der Waals interaction formed between helix A and IL-2Rβ, helix A and IL-2Rγ, or between helix C and IL-2Rβ,
optionally the insertion is occurred between I6 and S7, between S7 and D8, between D8 and L9, between L9 and K10, between K10 and K11, between K11 and I12, between H60 and D61, between D61 and T62, between E64 and N65, between N65 and L66, between I67 and I68, between I68 and L69, and/or between L69 and A70.

44. The IL-15 variant of claim 43, wherein the inserted amino acid is a non-polar amino acid such as Ala, Gly, Val, Leu, Ile, Met, Trp, Phe, Pro, a polar amino acid such as Ser, Thr, Cys, Tyr, Asn, Gln, a charged amino acid such as Asp, Glu, Lys, Arg, His, or an unnatural amino acid.

45. A fusion protein, wherein the fusion protein comprises the IL-15 variant of claim 39 operably linked to a non-IL-15 moiety, optionally the non-IL-15 moiety is an antigen-binding domain or an Fc domain.

46. A heterodimeric fusion protein comprising:
(A) a first chain, comprising an IL-15 domain operably linked to one chain of an Fc domain, wherein the IL-15 domain comprises the IL-15 variant of claim 39; and
(B) a second chain, comprising an IL-15Rα domain operably linked to the other chain of the Fc domain.

47. The heterodimeric fusion protein of claim 46, wherein the IL-15Rα domain comprises or consists of a wild-type IL-15Rα protein, an IL-15Rα variant or any fragment thereof that retains IL-15 binding activity, such as a fragment as set forth in SEQ ID No: 2.

48. The heterodimeric fusion protein of claim 46, wherein the Fc domain comprises a human IgG Fc such as a human IgG1 Fc or IgG2 Fc, or IgG3 Fc, or IgG4 Fc, or a variant thereof.

49. The heterodimeric fusion protein of claim 48, wherein each chain of the Fc variant comprises one or more substitutions compared to wild type human Fc to promote heterodimerization.

50. The heterodimeric fusion protein of claim 48, wherein the IL-15 domain and/or IL-15Rα domain is operably linked to the Fc domain via a linker.

51. The heterodimeric fusion protein of claim 46, wherein the first chain comprises an amino acid sequence as set forth in SEQ ID No: 7, 9, 11, 13, 15, 17, 19, 21, 23, 25, 27, 29, 31, 33, or 35, and/or the second chain comprises an amino acid sequence as set forth in SEQ ID No: 8, 10, 12, 14, 16, 18, 20, 22, 24, 26, 28, 30, 32, 34, or 36.

52. A nucleic acid molecule, comprising a nucleic acid sequence encoding the IL-15 variant of claim 39.

53. A vector comprising the nucleic acid molecule of claim 52.

54. A host cell comprising the vector of claim 53.

55. A pharmaceutical composition comprising the heterodimeric fusion protein of claim 46 or a nucleic acid molecule encoding the heterodimeric fusion protein, and a pharmaceutically acceptable carrier.

56. A method for producing the heterodimeric fusion protein of claim 46 comprising the steps of:
expressing the heterodimeric fusion protein in a host cell comprising a vector(s) encoding one or both chains of the heterodimeric fusion protein; and
isolating the heterodimeric fusion protein from the host cell culture.

57. A method for treating or preventing cancer or infectious diseases in a subject, comprising administering an effective amount of the heterodimeric fusion protein of claim 46 to the subject, optionally further comprising administering an additional anti-tumor therapy, such as cell immunotherapy including tumor-infiltrating lymphocyte (TIL) therapy, T cell receptor (TCR) therapy, chimeric antigen receptor (CAR) T cell therapy, and NK cell therapy, as well as targeted therapy and chemotherapy.

58. The method of claim 57, wherein the cancer is selected from breast cancer, lung cancer, colon cancer, ovarian cancer, melanoma, bladder cancer, renal cell carcinoma, liver cancer, prostate cancer, stomach cancer, pancreatic cancer, NSCLC, non-Hodgkin's lymphoma, chronic lymphocytic leukemia, diffuse large B-cell lymphoma, and multiple myeloma.

59. A kit, comprising a container comprising the heterodimeric fusion protein of claim 46.


SEQUENCE LISTING (36 sequences)

SEQ ID NO: 1 - protein, 114 aa, homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 65 aa, homo sapiens
ITCPPPMSVEHADIWVKSYSLYSRERYICNSGFKRKAGTSSLTECVLNKATNVAHWTTPSLKCIR

SEQ ID NO: 3 - protein, 231 aa, Artificial sequence
  Fc domain one chain (V47)
EPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 231 aa, Artificial sequence
  Fc domain the other chain (V47)
EPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 5 - protein, 231 aa, Artificial sequence
  Fc domain, one chain (V42)
EPKSSDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 231 aa, Artificial sequence
  Fc domain, the other chain (V42)
EPKSSDKTHTCPPCPAPELLRGPSVFLFPPKPKDTLMISRTPEVTCVVVDVSHEDPEVKFNWYVDGVEVHNAKTKPREEQYNSTYRVVSVLTVLHQDWLNGKEYKCKVSNKARPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 7 - protein, 350 aa, Artificial sequence
  V0005-V47, one chain
NWVNVISELKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 301 aa, Artificial sequence
  V0005-V47, the other chain
ITCPPPMSVEHADIWVKSYSLYSRERYICNSGFKRKAGTSSLTECVLNKATNVAHWTTPSLKCIRGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 9 - protein, 350 aa, Artificial sequence
  V0006-V47, one chain
NWVNVISSLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 301 aa, Artificial sequence; identical to SEQ ID NO: 8
  V0006-V47, the other chain

SEQ ID NO: 11 - protein, 350 aa, Artificial sequence
  V0015-V47, one chain
NWVNVISDLKKIEDLIQSMHIDATLYTESDVHPSCKVTAMKCFLLELQVISLESGDASIHDTVEQLIILANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 301 aa, Artificial sequence; identical to SEQ ID NO: 8
  V0015-V47, the other chain

SEQ ID NO: 13 - protein, 350 aa, Artificial sequence
  V0018-V47, one chain
NWVNVISDLKKIEDLIQSMHIDATLYTESDVHPSCKVTAMKCFLLELQVISLESGDASIHDTVENLIIAANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 301 aa, Artificial sequence; identical to SEQ ID NO: 8
  V0018-V47, the other chain

SEQ ID NO: 15 - protein, 350 aa, Artificial sequence
  V0020-V47, one chain
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVNMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 301 aa, Artificial sequence; identical to SEQ ID NO: 8
  V0020-V47, the other chain

SEQ ID NO: 17 - protein, 350 aa, Artificial sequence
  V0058-V47, one chain
NWVNVINDLQKIEDLIQSMHIDATLYTESDVHPSCKVTAMKCFLLELQVISLESGDASIHDTVENLIALANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 301 aa, Artificial sequence; identical to SEQ ID NO: 8
  V0058-V47, the other chain

SEQ ID NO: 19 - protein, 350 aa, Artificial sequence
  V0061-V47, one chain
NWVNVINDLKKIEDLIQSMHIDATLYTESDVHPSCKVTAMKCFLLELQVISLESGDASIHDTVENLIAVANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 301 aa, Artificial sequence; identical to SEQ ID NO: 8
  V0061-V47, the other chain

SEQ ID NO: 21 - protein, 350 aa, Artificial sequence
  V0063-V47, one chain
NWVNVINDLKKIEDLIQSMHIDATLYTESDVHPSCKVTAMKCFLLELQVISLESGDASIHDTVENLIALANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 301 aa, Artificial sequence; identical to SEQ ID NO: 8
  V0063-V47, the other chain

SEQ ID NO: 23 - protein, 350 aa, Artificial sequence
  V0067-V47, one chain
NWVNVISDLKKIEDLIQSMHIDATLYTESDVHPSCKVTAMKCFLLELQVISLESGDASIHDTVENLIAVANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 301 aa, Artificial sequence; identical to SEQ ID NO: 8
  V0067-V47, the other chain

SEQ ID NO: 25 - protein, 351 aa, Artificial Sequence
  V0001.uIgG1V805, one chain
NWVNVISADLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 301 aa, Artificial sequence; identical to SEQ ID NO: 8
  V0001.uIgG1V805, other chain

SEQ ID NO: 27 - protein, 351 aa, Artificial sequence
  V0002.uIgG1V805, one chain
NWVNVISDALKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 301 aa, Artificial sequence; identical to SEQ ID NO: 8
  V0002.uIgG1V805, other chain

SEQ ID NO: 29 - protein, 351 aa, Artificial sequence
  V0003.uIgG1V805, one chain
NWVNVISDLAKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 301 aa, Artificial sequence; identical to SEQ ID NO: 8
  V0003.uIgG1V805, other chain

SEQ ID NO: 31 - protein, 351 aa, Artificial sequence
  V0004.uIgG1V805, one chain
NWVNVISDLKAKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 301 aa, Artificial sequence; identical to SEQ ID NO: 8
  V0004.uIgG1V805, other chain

SEQ ID NO: 33 - protein, 351 aa, Artificial sequence
  V0005.uIgG1V805, one chain
NWVNVISDLKKIEDLIQSMHIDATLYTESDVHPSCKVTAMKCFLLELQVISLESGDASIHDTVEANLIILANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 301 aa, Artificial sequence; identical to SEQ ID NO: 8
  V0005.uIgG1V805, other chain

SEQ ID NO: 35 - protein, 351 aa, Artificial sequence
  V0006.uIgG1V805, one chain
NWVNVISDLKKIEDLIQSMHIDATLYTESDVHPSCKVTAMKCFLLELQVISLESGDASIHDTVENALIILANNSLSSNGNVTESGCKECEELEEKNIKEFLQSFVHIVQMFINTSGGGGSEPKSSDKTHTCPPCPAPELLRGPSVFLFPPKPKDTLYITREPEVTCVVVDVSHEDPEVKFNWYVDGVEVHNAKTKPREEQYNSTYRVVSVLTVLHQDWLNGKEYKCKVSNKARPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 301 aa, Artificial sequence; identical to SEQ ID NO: 8
  V0006.uIgG1V805, other chain